(19) United States
(12) Patent Application Publication
(10) Pub. No.: US 2026/0042838 A1
(43) Pub. Date: Feb. 12, 2026

(54) METHODS AND COMPOSITIONS FOR TREATING AUTOIMMUNE HEMOLYTIC ANEMIA (AIHA)

(71) Applicant: Zenas BioPharma, Inc., Waltham, MA (US)

(72) Inventors: Allen POMA, Waltham, MA (US); Xiaodong WANG, Waltham, MA (US); Shauna QUINN, Waltham, MA (US)

(73) Assignee: Zenas BioPharma, Inc., Waltham, MA (US)

(21) Appl. No.: 19/367,474

(22) Filed: Oct. 23, 2025

Related U.S. Application Data
(63) Continuation of application No. PCT/US2024/025810, filed on Apr. 23, 2024.
(60) Provisional application No. 63/497,936, filed on Apr. 24, 2023.

(51) Int. Cl. C07K 16/28; A61K 31/573; A61K 39/00; A61P 7/06; A61P 37/06

(52) U.S. Cl. CPC C07K 16/2803; A61K 31/573; A61P 7/06; A61P 37/06; C07K 16/283; A61K 2039/54; A61K 2039/545

(57) ABSTRACT

The present invention provides compositions and methods of treating and improving the symptoms of AIHA using an antibody or antigen-binding fragment thereof that specifically binds human CD19.

### CROSS REFERENCE TO RELATED APPLICATIONS

The present application is a bypass continuation of International Application No. PCT/US2024/025810, filed on Apr. 23, 2024, which claims benefit of priority to U.S. Provisional Patent Application No. 63/497,936, filed on Apr. 24, 2023, all of which are hereby incorporated by reference in their entireties.

### SEQUENCE LISTING

The instant application contains a Sequence Listing which has been submitted electronically in XML file format and is hereby incorporated by reference in its entirety. Said XML copy, created on Oct. 22, 2025, is named “ZEN-010US1_SequenceListing” and is 16,384 bytes.

### BACKGROUND

Autoimmune hemolytic anemia (AIHA) is an uncommon, acquired autoimmune disorder, in which autoantibodies directed against self-red blood cell (RBC) membrane antigens lead to accelerated destruction of RBCs. The destruction of the RBCs by immune cells more rapidly than the production rate of production of new cells, leads patients to develop anemia, generalized fatigue, dizziness, syncope, malaise, chest pressure/pain, cognitive dysfunction, weakness, a pale skin color (pallor), palpitations, shortness of breath (dyspnea), appearance of jaundice and abnormally dark urine (hematouria), usually suggestive of hemolysis. Murine models of AIHA show that reticulocytes are preferentially targeted by anti-RBC autoantibodies and an increase in oxidative stress may trigger autoantibody production. Though not usually fatal, most patients have a reduced health-related quality of life. Current therapies used for treatment of AIHA include steroids, immunosuppressants and splenectomy. Prolonged steroid use carries a significant risk for infection, diabetes, and fracture. Harnessing treatment and defining a risk-adapted therapy for AIHA (e.g., wAIHA) is an emerging unmet need.

### SUMMARY

Among other things, the present disclosure provides methods, compositions, and use of obexelimab for treating human patients with autoimmune hemolytic anemia (AIHA) and prevention of relapse. In one aspect, the present invention provides a method of treating autoimmune hemolytic anemia (AIHA), comprising administering obexelimab subcutaneously to a human patient at a dose of 250 mg once a week. In some embodiments, the present disclosure is directed to a method of treating of warm AIHA (wAIHA) using CD19 antibodies such as obexelimab.

In one aspect the present invention provides a method of treating AIHA, wherein the AIHA is categorized as warm AIHA. In some embodiments, the patient does not have cold agglutinin syndrome, cold AIHA, mixed AIHA or PCH.

In some aspects, the present disclosure encompasses a method of treating autoimmune hemolytic anemia (AIHA), comprising administering obexelimab subcutaneously to a human patient at a dose of 250 mg once a week. In some embodiments, the patient has a Hgb level of ≥7 to <10 g/dL.

In some embodiments, the patient has been diagnosed with warm autoimmune hemolytic anemia (wAIHA). In some embodiments, the patient has at least one sign or symptom of anemia. In some embodiments, the patient also has failed at least 1 prior wAIHA treatment regimen. In some embodiments, the prior wAIHA treatment regimen is GC (glucocorticoid) or immunosuppression therapy.

In some embodiments, the method comprises measuring Hgb and/or LDH levels. In some embodiments, the failure of the prior wAIHA treatment regimen comprises reduction in Hgb of ≥1 g/dL. In some embodiments, the failure of the prior wAIHA treatment regimen comprises an increase in LDH of ≥1.5× upper limit of normal (ULN). In some embodiments, obexelimab is administered concurrently with a GC therapy. In some embodiments, the GC therapy is administered at a dose of 20-60 mg/day prednisone or equivalent. In some embodiments, the GC therapy is administered at a dose of 1-1.5 mg/kg/day prednisone or equivalent.

In some embodiments, the patient maintains a Hgb level of ≥7 g/dL following administration of obexelimab. In some embodiments, the patient maintains a Hgb level of ≥7 g/dL, ≥8 g/dL, ≥9 g/dL or ≥10 g/dL. In some embodiments, the patient achieves a Hgb≥10 g/dL. In some embodiments, the patient achieves a Hgb≥2 g/dL increase compared to Hgb levels prior to treatment with obexelimab. In some embodiments, obexelimab is administered for a time period sufficient to improve, stabilize or reduce one or more symptoms of wAIHA relative to a control.

In some embodiments, the patient achieves improvement in Hgb after at least 4, 5, 6, 7, 8, 9, 10, 11, 12 weeks of obexelimab administration. In some embodiments, the patient achieves improvement in FACIT-F score compared to baseline (e.g., the FACIT-F score prior to treatment with obexelimab). In some embodiments, the patient achieves improvement in EQ-5D-5L index score from baseline (e.g., the Q-5D-5L index score prior to treatment with obexelimab). In some embodiments, the patient achieves one or more of the following: (a) decrease in circulating absolute T, B, and NK cell count; (b) decrease in Ig levels and ratios (e.g., IgG, IgM, IgA, IgE); (c) increase in CD19 target receptor occupancy; (d) decrease in reticulocyte count; (e) decrease in LDH; (f) increase in haptoglobin; (g) decrease in indirect bilirubin (unconjugated bilirubin) following administration obexelimab. In some embodiments, the human patient is relapsed or refractory to rituximab. In some embodiments, the patient≥18 years of age.

In some embodiments, the patient does not have cold autoimmune hemolytic amenia (CAD). In some embodiments, the patient does not have mixed type autoimmune hemolytic amenia. In some embodiments, the patient does not have paroxysmal cold hemoglobinuria (PCH).

In some embodiments, the obexelimab is administered in a liquid formulation comprising 125 mg/mL obexelimab, 2.35 mg/mL sodium acetate trihydrate, 0.17 mg/mL acetic acid, 30 mg/mL L-proline, 0.1 mg/mL polysorbate 80 at pH 5.5. In some embodiments, the obexelimab is administered as 2×1 mL injections or 1×2 mL injection. In some embodiments, the obexelimab is administered using a prefilled syringe or autoinjector.

### BRIEF DESCRIPTION OF DRAWING

FIG. 1 is a schematic showing an exemplary clinical study design described for treating wAIHA.

### DETAILED DESCRIPTION

The present invention provides, among other things, methods of treating wAIHA-related disease by administering to a human patient 18 years of age or older in need of treatment an anti-CD19 antibody at a therapeutically effective dose and an administration interval for a treatment period sufficient to improve, stabilize or reduce one or more symptoms of the wAIHA-related autoimmune disease relative to a control (e.g., start of treatment).

Various aspects of the invention are described in detail in the following sections. The use of sections is not meant to limit the invention. Each section can apply to any aspect of the invention. In this application, the use of “or” means “and/or” unless stated otherwise.

### Definitions

Described herein are several definitions. Such definitions are meant to encompass grammatical equivalents.

Antibody: The term “antibody” herein is meant a protein consisting of one or more polypeptides substantially encoded by all or part of the recognized immunoglobulin genes. The recognized immunoglobulin genes, for example in humans, include the kappa (κ), lambda (1), and heavy chain genetic loci, which together comprise the myriad variable region genes, and the constant region genes mu (u), delta (d), gamma (γ), sigma(s), and alpha (a) which encode the IgM, IgD, IgG (lgG1, lgG2, lgG3, and lgG4), IgE, and IgA (lgA1 and lgA2) isotypes respectively. Antibody herein is meant to include full length antibodies and antibody fragments, and may refer to a natural antibody from any organism, an engineered antibody, or an antibody generated recombinantly for experimental, therapeutic, or other purposes.

Baseline: The term “baseline” is defined as values of a parameter prior to commencement of treatment with a therapeutic. In some embodiments “baseline” is an initial measurement of a condition that is taken at an early time point and used for comparison over time to look for changes. In some embodiments, the baseline is time “zero”, before the participants in the study receive an experimental agent or intervention, or negative control; drug safety and efficacy may be determined by monitoring changes in baseline values.

CD32b⁺ cell or FcγRIIb⁺ cell: The terms “CD32b⁺ cell” or “FcγRIIb cell” as used herein is meant any cell or cell type that expresses CD32b (FcγRIIb). CD32b+ cells include but are not limited to B cells, plasma cells, dendritic cells, macrophages, neutrophils, mast cells, basophils, or eosinophils.

CDC or complement dependent cytotoxicity: The terms “CDC” or “complement dependent cytotoxicity” as used herein is meant the reaction wherein one or more complement protein components recognize bound antibody on a target cell and subsequently cause lysis of the target cell.

Effector Function: The term “effector function” as used herein is meant a biochemical event that results from the interaction of an antibody Fc region with an Fc receptor or ligand. Effector functions include FcγR-mediated effector functions such as ADCC and ADCP, and complement-mediated effector functions such as CDC. Further, effector functions include FcγRIIb-mediated effector functions, such as inhibitory functions (e.g., downregulating, reducing, inhibiting etc., B cell responses, e.g., a humoral immune response).

Effector Cell: The term “effector cell” as used herein is meant a cell of the immune system that expresses one or more Fc and/or complement receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and gd T cells, and may be from any organism including but not limited to humans, mice, rats, rabbits, and monkeys.

Fc or Fc region: The terms “Fc” or “Fc region,” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cgamma2 and Cgamma3 (Og2 and C 3) and the hinge between Cgammal (Ogî) and Cgamma2 (Cy2). Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an Fc polypeptide, as described below.

Fc gamma receptor, or FcγR: The terms “Fc gamma receptor” or “FcγR” as used herein is meant any member of the family of proteins that bind the IgG antibody Fc region and are substantially encoded by the FcγR genes. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V1 58 and F158) and FcγRIIIb (including allotypes FcγRIIIb-NA1 and FcγRIIIb-NA2) (Jefferis et al., 2002, Immunol Lett 82:57-65, incorporated entirely by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

Modification: The term “modification” herein is meant an alteration in the physical, chemical, or sequence properties of a protein, polypeptide, antibody, or immunoglobulin. Modifications described herein include amino acid modifications (including amino acid substitutions) and glycoform modifications.

Target Antigen: The term “target antigen” as used herein is meant the molecule that is bound by the variable region of a given antibody, or the fusion partner of an Fc fusion. A target antigen may be a protein, carbohydrate, lipid, or other chemical compound. An antibody or Fc fusion is said to be “specific” for a given target antigen based on having affinity for the target antigen. In some embodiments, the target antigen for the obexelimab is CD19.

Target cell: The term “target cell” as used herein is meant a cell that expresses a target antigen.

Oexelimab: The term “Obexelimab” as used herein, is an Fc engineered humanized monoclonal antibody (mAb) that binds to the human B-cell restricted surface antigen CD19 and has enhanced Fc binding to Fcγ receptor IIb (FcγRIIb). The molecule is an IgG1 immunoglobulin with a kappa light chain and 2 amino acid substitutions in the constant portion of the heavy chain. Obexelimab is a monoclonal antibody with a projected mass of approximately 147,426 Da based on the amino acid sequence. The heavy and light chains of obexelimab are given by SEQ ID NO: 10, and SEQ ID NO: 9, respectively.

Rescue Therapy: The term “rescue therapy” as used herein refers to use of a therapy to treat a suspected remerging disease or worsening of disease symptoms. In some embodiments, the rescue therapy is a different approved therapy. In some embodiments, the rescue therapy is any therapy that may be used to lessen the symptoms associated with wAIHA. In some embodiments, the rescue therapy for wAIHA is glucocorticoid (GC) rescue therapy. In some embodiments, the rescue therapy for wAIHA is a blood transfusion. Non-limiting examples of rescue therapies may include splenectomy, blood transfusion, rituximab, and other anti-CD19 antibody therapies.

### Obexelimab and Variants Thereof

According to the present invention, obexelimab or a variant thereof is used to treat a human patient suffering from an wAIHA. Obexelimab is a monoclonal antibody specific for CD19 comprising: a light chain comprising a variable region having:

a CDR1 comprising
(SEQ ID NO: 2)
RSSKSLQNVNGNTYLY,
a CDR2 comprising
(SEQ ID NO: 3)
RMSNLNS,
and
a CDR3 comprising
(SEQ ID NO: 4)
MQHLEYPIT;

and
a heavy chain comprising a variable region having

a CDR1 comprising
(SEQ ID NO: 5)
SYVMH,
a CDR2 comprising
(SEQ ID NO: 6)
WIGYINPYNDGTKY,
and
a CDR3 comprising
(SEQ ID NO: 7)
GTYYYGTRVFDY,

wherein the heavy chain comprises amino acid substitutions in the Fc region S267E and L328F as compared to SEQ ID NO: 8:

(SEQ ID NO: 8)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK,

wherein the numbering is according to the EU index, as in Kabat.

In an embodiment, obexelimab comprises: a light chain comprising an amino acid sequence of (SEQ ID NO: 9) and heavy chain comprising an amino acid sequence of, (SEQ ID NO: 10), as given by Table 1.

TABLE 1
Sequence of Heavy chain and Light chain amino acid
sequence of Obexelimab

| Heavy Chain | Light Chain |
|---|---|
| EVQLVESGGGLVKPGGSLKLSCAASGY | DIVMTQSPATLSLSPGERATLSCRSSKSLQN |
| TFTSYVMHWVRQAPGKGLEWIGYINPY | VNGNTYLYWFQQKPGQSPQLLIYRMSNLNS |
| NDGTKYNEKFQGRVTISSDKSISTAYME | GVPDRFSGSGSGTEFTLTISSLEPEDFAVYYC |
| LSSLRSEDTAMYYCARGTYYYGTRVFD | MQHLEYPITFGAGTKLEIKRTVAAPSVFIFPP |
| YWGQGTLVTVSSASTKGPSVFPLAPSSK | SDEQLKSGTASVVCLLNNFYPREAKVQWK |
| STSGGTAALGCLVKDYFPEPVTVSWNS | VDNALQSGNSQESVTEQDSKDSTYSLSSTLT |
| GALTSGVHTFPAVLQSSGLYSLSSVVTV | LSKADYEKHKVYACEVTHQGLSSPVTKSFN |
| PSSSLGTQTYICNVNHKPSNTKVDKKVE | RGEC (SEQ ID NO: 9) |
| PKSCDKTHTCPPCPAPELLGGPSVFLFPP | |
| KPKDTLMISRTPEVTCVVVDVEHEDPE | |
| VKFNWYVDGVEVHNAKTKPREEQYNS | |
| TYRVVSVLTVLHQDWLNGKEYKCKVS | |
| NKAFPAPIEKTISKAKGQPREPQVYTLPP | |
| SREEMTKNQVSLTCLVKGFYPSDIAVE | |
| WESNGQPENNYKTTPPVLDSDGSFFLY | |
| SKLTVDKSRWQQGNVFSCSVMHEALH | |
| NHYTQKSLSLSPGK (SEQ ID NO: 10) | |

Variable region

| | |
|---|---|
| EVQLVESGGGLVKPGGSLKLSCAASGY | DIVMTQSPATLSLSPGERATLSCRSSKSLQN |
| TFTSYVMHWVRQAPGKGLEWIGYINPY | VNGNTYLYWFQQKPGQSPQLLIYRMSNLNS |
| NDGTKYNEKFQGRVTISSDKSISTAYME | GVPDRFSGSGSGTEFTLTISSLEPEDFAVYYC |
| LSSLRSEDTAMYYCARGTYYYGTRVFD | MQHLEYPITFGAGTKLEIK (SEQ ID NO: 11) |
| YWGQGTLVTVSS (SEQ ID NO: 12) | |

In one embodiment, Obexelimab comprises a light chain variable region comprising SEQ ID NO: 11 and a heavy chain variable region comprising SEQ ID NO: 12.

Obexelimab works by exploiting the regulation of B-cell receptor (BCR) signaling by FcγRIIb1. Obexelimab binds CD19 of the BCR complex and its Fc is engineered to increase its affinity for the inhibitory FcγRIIb. Since CD19 is associated with the BCR, Obexelimab tethering of CD19 to FcγRIIb on the same cell poises the BCR complex for inhibition upon antigen-induced BCR aggregation. Obexelimab capitalizes upon the natural inhibitory mechanism of FcγRIIb, the only Fc receptor expressed by B cells, which acts as a negative regulator in conditions of antigen excess and immune complex formation (Chu et al., 2014). Obexelimab may also have an improved safety profile compared to B cell depleting antibodies as it does not mediate B cell killing.

### Variants

In an embodiment, a variant of obexelimab is an immunoglobulin specific for CD19 comprises: a light chain comprising a variable region having a CDR1 comprising SEQ ID NO: 2, a CDR2 comprising SEQ ID NO: 3, and a CDR3 comprising SEQ ID NO: 4; and a heavy chain comprising a variable region having a CDR1 comprising SEQ ID NO: 5, a CDR2 comprising SEQ ID NO: 6, and a CDR3 comprising SEQ ID NO: 7, wherein the heavy chain comprises amino acid substitutions in the Fc region S267E and L328F as compared to SEQ ID NO: 8, wherein the numbering is according to the EU index, as in Kabat.

In some embodiments, a variant of obexelimab comprises a heavy chain variable region (VH) and/or a light chain variable region (VL), comprising a CDR1, a CDR2, and a CDR3, each of which differs by no more than 1, 2, 3, 4 or 5 amino acid residues from each of SEQ ID NO: 2, SEQ ID NO: 3, SEQ ID NO: 4, SEQ ID NO: 5, SEQ ID NO: 6 and/or SEQ ID NO: 7.

In some embodiments, the variant of obexelimab comprises: a light chain variable region comprising an amino acid sequence that is at least 90%, 91%, 92%, 93%, 94%, 95%, 96%, 97%, 98% or 99% identical to SEQ ID NO: 11. In some embodiments, the variant of obexelimab comprises: a heavy chain variable region comprising an amino acid sequence that is at least 90%, 91%, 92%, 93%, 94%, 95%, 96%, 97%, 98% or 99% identical to SEQ ID NO: 12. In some embodiments, the variant of obexelimab comprises: a light chain variable region comprising an amino acid sequence that is at least 90%, 91%, 92%, 93%, 94%, 95%, 96%, 97%, 98% or 99% identical to SEQ ID NO: 11 and a heavy chain variable region comprising an amino acid sequence that is at least 90%, 91%, 92%, 93%, 94%, 95%, 96%, 97%, 98% or 99% identical to SEQ ID NO: 12, and further comprises amino acid substitutions in the Fc region S267E and L328F as compared to SEQ ID NO: 8, wherein the numbering is according to the EU index.

In an embodiment, the variant of obexelimab comprises: a light chain comprising an amino acid sequence 70%, 75%, 80%, 85%, 90%, 95%, 98% or 99% identical to SEQ ID NO: 9. In an embodiment, the variant of obexelimab comprises: a heavy chain comprising an amino acid sequence 70%, 75%, 80%, 85%, 90%, 95%, 98% or 99% identical to SEQ ID NO: 10.

In some embodiments, the variant of obexelimab comprises: a light chain comprising an amino acid sequence that is at least 90%, 91%, 92%, 93%, 94%, 95%, 96%, 97%, 98% or 99% identical to SEQ ID NO: 9 and a heavy chain comprising an amino acid sequence that is at least 90%, 91%, 92%, 93%, 94%, 95%, 96%, 97%, 98% or 99% identical to SEQ ID NO: 10, and the heavy chain of the variant comprises amino acid substitutions in the Fc region S267E and L328F as compared to SEQ ID NO: 8 wherein the numbering is according to the EU index, as in Kabat.

In an embodiment, the variant of obexelimab comprises: a light chain comprising an amino acid sequence 70%, 75%, 80%, 85%, 90%, 95%, 98% or 99% identical to SEQ ID NO: 9 and/or amino acid substitutions in the Fc region S267E and L328F as compared to SEQ ID NO: 10, wherein the numbering is according the EU index, as in Kabat.

In some embodiments, a suitable variant of obexelimab binds to the same epitope on human CD19, as an antibody comprising a light chain of SEQ ID NO: 9 and a heavy chain of SEQ ID NO: 10. Epitope binding may be determined by a method known in the art.

In some embodiments, a suitable variant of obexelimab competes for binding to human CD19, as an antibody comprising a light chain of SEQ ID NO: 9 and a heavy chain of SEQ ID NO: 10, under a binning assay known in the art. As used herein, a binning assay refers to any method to regionally map the epitope to which the antibody binds. Standard methods for such antibody characterization, also known as epitope binning, typically involve surface plasmon resonance (SPR) technology. Using SPR, monoclonal antibody candidates are screened pairwise for binding to a target protein. Other standard methods involve ELISA-based screens and may require synthesis of sets of overlapping peptides corresponding to the protein of interest.

In some embodiments, a suitable variant of obexelimab binds to the extracellular domain of human CD19, wherein the human CD19 comprises an amino acid sequence of SEQ ID NO: 1:

(SEQ ID NO: 1)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR

### Fc Receptor Binding Properties

Anti-CD19 antibodies (e.g., obexelimab) disclosed herein comprise an Fc variant that has enhanced Fc binding to the inhibitory Fcγ receptor IIb (FcγRIIb). FcγRIIb, the only FcR on B cells, serves as an antibody-sensing down-regulator of humoral immunity that is naturally engaged by immune complexes. When sufficient antibody is raised against a given antigen, specific immune complexes form and co-engage FcγRIIb and the B cell receptor (BCR) with high avidity, selectively suppressing only B cells recognizing cognate antigen. In addition, FcγRIIb regulates the activity of other B cell stimulators including interleukin (IL)-4, LPS, and BAFF that amplify BCR-driven proliferation and differentiation. By simultaneously binding CD19 and FcγRIIb, obexelimab (and variants described herein) mimics the action of antigen-antibody complexes and down-regulates B cell activity.

The Fc variants disclosed herein may be optimized for a variety of Fc receptor binding properties. An Fc variant that is engineered or predicted to display one or more optimized properties is herein referred to as an “optimized Fc variant.” Properties that may be optimized include but are not limited to enhanced or reduced affinity for an FcγR. In one embodiment, the Fc variants disclosed herein are optimized to possess enhanced affinity for an inhibitory receptor FcγRIIb. In other embodiments, immunoglobulins disclosed herein provide enhanced affinity for FcγRIIb, yet reduced affinity for one or more activating FcγRs, including for example FcγRI, FcγRIIa, FcγRIIIa, and/or FcγRIIIb. The FcγR receptors may be expressed on cells from any organism, including but not limited to human, cynomolgus monkeys, and mice. The Fc variants disclosed herein may be optimized to possess enhanced affinity for human FcγRIIb.

An Fc variant comprises one or more amino acid modifications relative to a parent Fc polypeptide, wherein the amino acid modification(s) provide one or more optimized properties. An Fc variant disclosed herein differs in amino acid sequence from its parent by virtue of at least one amino acid modification. Thus, Fc variants disclosed herein have at least one amino acid modification compared to the parent. Alternatively, the Fc variants disclosed herein may have more than one amino acid modification as compared to the parent, for example from about two to fifty amino acid modifications, e.g., from about two to ten amino acid modifications, from about two to about five amino acid modifications, etc. compared to the parent. Thus, the sequences of the Fc variants and those of the parent Fc polypeptide are substantially homologous. For example, the variant Fc variant sequences herein will possess about 80% homology with the parent Fc variant sequence, e.g., at least about 90% homology, at least about 95% homology, at least about 98% homology, at least about 99% homology, etc. Modifications disclosed herein include amino acid modifications, including insertions, deletions, and substitutions. Modifications disclosed herein also include glycoform modifications.

Modifications may be made genetically using molecular biology or may be made enzymatically or chemically.

Fc variants disclosed herein are defined according to the amino acid modifications that compose them. Thus, for example, S267E is an Fc variant with the substitution S267E relative to the parent Fc polypeptide. Likewise, S267E/L328F defines an Fc variant with the substitutions S267E and L328F relative to the parent Fc polypeptide. The identity of the WT amino acid may be unspecified, in which case the aforementioned variant is referred to as 267E/328F. It is noted that the order in which substitutions are provided is arbitrary, that is to say that, for example, 267E/328F is the same Fc variant as 328F/267E, and so on. Unless otherwise noted, positions discussed herein are numbered according to the EU index as described in Kabat (Kabat et al., 1991, Sequences of Proteins of Immunological Interest, 5th Ed., United States Public Health Service, National Institutes of Health, Bethesda, hereby entirely incorporated by reference). In brief, EU is the name of the first antibody molecule whose entire amino acid sequence was determined (Edelman et al., 1969, Proc Natl Acad Sci USA 63:78-85, hereby entirely incorporated by reference), and its amino acid sequence has become the standard numbering scheme for heavy chain constant regions. The EU protein has become the standard reference for defining numbering. Kabat et al. lists the EU sequence in a set of indices aligning it with other antibody sequences, serving as a necessary tool for aligning antibodies to the EU numbering scheme. Thus, as appreciated by those of skill in the art, the standard way of referencing the EU numbering is to refer to Kabat et al.'s alignment of sequences, because it puts EU in context with antibodies of other variable domain lengths. As such, as used herein, “the EU index as in Kabat” or “numbering is according to the EU index, as in Kabat” refers to the numbering of the EU antibody as described in Kabat.

In certain embodiments, the Fc variants disclosed herein are based on human IgG sequences, and thus human IgG sequences are used as the “base” sequences against which other sequences are compared, including but not limited to sequences from other organisms, for example rodent and primate sequences. Immunoglobulins may also comprise sequences from other immunoglobulin classes such as IgA, IgE, IgGD, IgGM, and the like. It is contemplated that, although the Fc variants disclosed herein are engineered in the context of one parent IgG, the variants may be engineered in or “transferred” to the context of another, second parent IgG. This is done by determining the “equivalent” or “corresponding” residues and substitutions between the first and second IgG, typically based on sequence or structural homology between the sequences of the first and second IgGs. To establish homology, the amino acid sequence of a first IgG outlined herein can be directly compared to the sequence of a second IgG. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first immunoglobulin are defined. Alignment of conserved residues may conserve 100% of such residues. However, alignment of greater than 75% or as little as 50% of conserved residues is also adequate to define equivalent residues. Equivalent residues may also be defined by determining structural homology between a first and second IgG that is at the level of tertiary structure for IgGs whose structures have been determined. In this case, equivalent residues are defined as those for which the atomic coordinates of two or more of the main chain atoms of a particular amino acid residue of the parent or precursor (N on N, CA on CA, Con C and O on O) are within about 0.13 nm, after alignment. In another embodiment, equivalent residues are within about 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent IgG in which the IgGs are made, what is meant to be conveyed is that the Fc variants discovered as disclosed herein may be engineered into any second parent IgG that has significant sequence or structural homology with the Fc variant. Thus, for example, if a variant antibody is generated wherein the parent antibody is human lgG1, by using the methods described above or other methods for determining equivalent residues, the variant antibody may be engineered in another lgG1 parent antibody that binds a different antigen, a human lgG2 parent antibody, a human IgA parent antibody, a mouse lgG2a or lgG2b parent antibody, and the like. Again, as described above, the context of the parent Fc variant does not affect the ability to transfer the Fc variants disclosed herein to other parent IgGs.

The term “greater affinity” or “improved affinity” or “enhanced affinity” or “better affinity” than a parent Fc polypeptide, as used herein is meant that an Fc variant binds to an Fc receptor with a significantly higher equilibrium constant of association (KA or Ka) or lower equilibrium constant of dissociation (KD or Kd) than the parent Fc polypeptide when the amounts of variant and parent polypeptide in the binding assay are essentially the same. For example, the Fc variant with improved Fc receptor binding affinity may display from about 5 fold to about 1000 fold, e.g. from about 10 fold to about 500 fold improvement in Fc receptor binding affinity compared to the parent Fc polypeptide, where Fc receptor binding affinity is determined, for example, by the binding methods disclosed herein, including but not limited to Biacore, by one skilled in the art. Accordingly, by “reduced affinity” as compared to a parent Fc polypeptide as used herein is meant that an Fc variant binds an Fc receptor with significantly lower KA or higher KD than the parent Fc polypeptide. Greater or reduced affinity can also be defined relative to an absolute level of affinity. For example, according to the data herein, WT (native) lgG1 binds FcγRIIb with an affinity of about 1.5 mM, or about 1500 nM. Furthermore, some Fc variants described herein bind FcγRIIb with an affinity about 10-fold greater to WT lgG1. As disclosed herein, greater or enhanced affinity means having a KD lower than about 100 nM, for example between about 10 nM-about 100 nM, between about 1-about 100 nM, or less than about 1 nM.

In one embodiment, the Fc variants provide selectively enhanced affinity to FcγRIIb relative to one or more activating receptors. Selectively enhanced affinity means either that the Fc variant has improved affinity for FcγRIIb relative to the activating receptor(s) as compared to the parent Fc polypeptide but has reduced affinity for the activating receptor(s) as compared to the parent Fc polypeptide, or it means that the Fc variant has improved affinity for both FcγRIIb and activating receptor(s) as compared to the parent Fc polypeptide, however the improvement in affinity is greater for FcγRIIb than it is for the activating receptor(s). In alternate embodiments, the Fc variants reduce or ablate binding to one or more activating FcγRs, reduce or ablate binding to one or more complement proteins, reduce or ablate one or more FcγR-mediated effector functions, and/or reduce or ablate one or more complement-mediated effector functions.

The presence of different polymorphic forms of FcγRs provides yet another parameter that impacts the therapeutic utility of the Fc variants disclosed herein. Whereas the specificity and selectivity of a given Fc variant for the different classes of FcγRs significantly affects the capacity of an Fc variant to target a given antigen for treatment of a given disease, the specificity or selectivity of an Fc variant for different polymorphic forms of these receptors may in part determine which research or pre-clinical experiments may be appropriate for testing, and ultimately which patient populations may or may not respond to treatment. Thus, the specificity or selectivity of Fc variants disclosed herein to Fc receptor polymorphisms, including but not limited to FcγRIIa, FcγRIIIa, and the like, may be used to guide the selection of valid research and pre-clinical experiments, clinical trial design, patient selection, dosing dependence, and/or other aspects concerning clinical trials.

Fc variants disclosed herein may comprise modifications that modulate interaction with Fc receptors other than FcγRs, including but not limited to complement proteins, FcRn, and Fc receptor homologs (FcRHs). FcRHs include but are not limited to FcRFH, FcRH2, FcRH3, FcRH4, FcRH5, and FcRH6 (Davis et al., 2002, Immunol. Reviews 190:123-136).

An important parameter that determines the most beneficial selectivity of a given Fc variant to treat a given disease is the context of the Fc variant. Thus, the Fc receptor selectivity or specificity of a given Fc variant will provide different properties depending on whether it composes an antibody, Fc fusion, or Fc variants with a coupled fusion partner. In one embodiment, an Fc receptor specificity of the Fc variant disclosed herein will determine its therapeutic utility. The utility of a given Fc variant for therapeutic purposes will depend on the epitope or form of the target antigen and the disease or indication being treated. For some targets and indications, greater FcγRIIb affinity and reduced activating FcγR-mediated effector functions may be beneficial. For other target antigens and therapeutic applications, it may be beneficial to increase affinity for FcγRIIb, or increase affinity for both FcγRIIb and activating receptors.

### Formulation and Pharmaceutical Compositions

The present invention provides pharmaceutical compositions and formulations of anti-CD19 antibodies (e.g., obexelimab). Formulations of the anti-CD19 antibody disclosed herein are prepared for storage by mixing said antibody having the desired degree of purity with optional pharmaceutically acceptable carriers, excipients or stabilizers (Remington's Pharmaceutical Sciences 16th edition, Osol, A. Ed., 1980, incorporated entirely by reference), in the form of lyophilized formulations or aqueous solutions.

In some embodiments, pharmaceutical compositions of interest comprise an anti-CD19 antibody (e.g., obexelimab) at various concentrations. In some embodiments, suitable formulations may comprise the antibody of interest at a concentration up to about 250 mg/ml (e.g., up to about 225 mg/ml, up to 200 mg/ml, up to 150 mg/ml, up to 140 mg/ml, up to 130 mg/ml, up to 125 mg/ml, up to 120 mg/ml, up to 115 mg/ml, up to 110 mg/ml, up to 105 mg/ml, up to 100 mg/ml, up to 90 mg/ml, up to 80 mg/ml, up to 70 mg/ml, up to 60 mg/ml, up to 50 mg/ml, up to 40 mg/ml, up to 30 mg/ml, up to 25 mg/ml, up to 20 mg/ml, up to 10 mg/ml).

In some embodiments, suitable formulations may contain the anti-CD19 antibody at a concentration ranging between about 10-300 mg/ml (e.g., about 10-250 mg/ml, about 10-200 mg/ml, about 10-180 mg/ml, about 10-160 mg/ml, about 10-150 mg/ml, about 10-140 mg/ml, about 10-130 mg/ml, about 10-125 mg/ml, about 100-125 mg/ml, about 100-180 mg/ml, about 100-150 mg/ml, about 100-130 mg/ml, about 100-125 mg/ml, about 100-170 mg/ml, about 100-160 mg/ml, about 100-150 mg/ml, about 100-200 mg/ml, about 120-130 mg/ml).

In some embodiments, formulations suitable for subcutaneous administration may contain a protein of interest at a concentration of approximately 100 mg/ml, 115 mg/ml, 120 mg/ml, 125 mg/ml, 130 mg/ml, 135 mg/ml, 140 mg/ml, 145 mg/ml, 150 mg/ml, 200 mg/ml or 300 mg/ml.

In some embodiments, isotonic solutions are used. In some embodiments, slightly hypertonic solutions (e.g., up to 300 mM (e.g., up to 250 mM, 200 mM, 175 mM, 150 mM, 125 mM) sodium chloride in 5 mM sodium phosphate at pH 7.0) and sugar-containing solutions (e.g., up to 3% (e.g., up to 2.4%, 2.0%, 1.5%, 1.0%) sucrose in 5 mM sodium phosphate at pH 7.0). In some embodiments, a suitable formulation composition is saline (e.g., 150 mM NaCl in water).

Many therapeutic agents, and in particular the antibodies of the present invention, require controlled pH and specific excipients to maintain their solubility and stability in the pharmaceutical compositions of the present invention.

The pH of the pharmaceutical composition is an additional factor which is capable of altering the solubility of an anti-CD19 antibody (e.g., obexelimab) in an aqueous pharmaceutical composition. In some embodiments, pharmaceutical compositions of the present invention contain one or more buffers. In some embodiments, compositions according to the invention contain an amount of buffer sufficient to maintain the optimal pH of said composition between about 4.0-8.0, between about 5.0-7.5, between about 5.5-7.0, between about 6.0-7.0 and between about 6.0-7.5. In other embodiments, the buffer comprises up to about 50 mM (e.g., up to about 45 mM, 40 mM, 35 mM, 30 mM, 25 mM, 20 mM, 15 mM, 10 mM, 5 mM) of sodium phosphate. Suitable buffers include, for example acetate, succinate, citrate, phosphate, other organic acids and tris(hydroxymethyl)aminomethane (“Tris”).

Suitable buffer concentrations can be from about 1 mM to about 100 mM, or from about 3 mM to about 20 mM, depending, for example, on the buffer and the desired isotonicity of the formulation. In some embodiments, a suitable buffering agent is present at a concentration of approximately 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, formulations contain an isotonicity agent to keep the formulations isotonic. Exemplary isotonicity agents include, but are not limited to, glycine, sorbitol, mannitol, sodium chloride and arginine. In some embodiments, suitable isotonic agents may be present in formulations at a concentration from about 0.01-5% (e.g., 0.05, 0.1, 0.15, 0.2, 0.3, 0.4, 0.5, 0.75, 1.0, 1.25, 1.5, 2.0, 2.5, 3.0, 4.0 or 5.0%) by weight.

In some embodiments, formulations may contain a stabilizing agent to protect the antibody. Typically, a suitable stabilizing agent is a non-reducing sugar such as sucrose, raffinose, trehalose, or amino acids such as glycine, arginine and methionine. The amount of stabilizing agent in a formulation is generally such that the formulation will be isotonic. However, hypertonic formulations may also be suitable. In addition, the amount of stabilizing agent must not be too low such that an unacceptable amount of degradation/aggregation of the antibody occurs. Exemplary stabilizing agent concentrations in the formulation may range from about 1 mM to about 400 mM (e.g., from about 30 mM to about 300 mM, and from about 50 mM to about 100 mM), or alternatively, from 0.1% to 15% (e.g., from 1% to 10%, from 5% to 15%, from 5% to 10%) by weight. In some embodiments, the ratio of the mass amount of the stabilizing agent and the therapeutic agent is about 1:1. In other embodiments, the ratio of the mass amount of the stabilizing agent and the therapeutic agent can be about 0.1:1, 0.2:1, 0.25:1, 0.4:1, 0.5:1, 1:1, 2:1, 2.6:1, 3:1, 4:1, 5:1, 10:1, or 20:1. In some embodiments, suitable for lyophilization, the stabilizing agent is also a lyoprotectants.

The pharmaceutical compositions, formulations and related methods of the invention are useful for delivering anti-CD19 antibodies (e.g., subcutaneously) and for the treatment of the associated diseases. The pharmaceutical compositions of the present invention are particularly useful for delivering anti-CD19 antibodies (e.g., obexelimab) to patients suffering from wAIHA.

In some embodiments, it is desirable to add a surfactant to formulations. Exemplary surfactants include nonionic surfactants such as Polysorbates (e.g., Polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristarnidopropyl-, palmidopropyl-, or isostearamidopropyl-dimethylamine; sodium methyl cocoyl-, or disodium methyl of eyl-taurate; and the MONAQUAT™ series (Mona Industries, Inc., Paterson, N.J.), polyethyl glycol, polypropyl glycol, and copolymers of ethylene and propylene glycol (e.g., Pluronics, PF68, etc). Typically, the amount of surfactant added is such that it reduces aggregation of the protein and minimizes the formation of particulates or effervescences. For example, a surfactant may be present in a formulation at a concentration from about 0.001-0.5% (e.g., about 0.005-0.05%, or 0.005-0.01%). In particular, a surfactant may be present in a formulation at a concentration of approximately 0.005%, 0.01%, 0.02%, 0.1%, 0.2%, 0.3%, 0.4%, or 0.5%, etc.

In some embodiments, suitable formulations may further include one or more bulking agents, in particular, for lyophilized formylations. A “bulking agent” is a compound which adds mass to the lyophilized mixture and contributes to the physical structure of the lyophilized cake. For example, a bulking agent may improve the appearance of lyophilized cake (e.g., essentially uniform lyophilized cake). Suitable bulking agents include, but are not limited to, sodium chloride, lactose, mannitol, glycine, sucrose, trehalose, hydroxyethyl starch. Exemplary concentrations of bulking agents are from about 1% to about 10% (e.g., 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, and 10.0%).

Formulations in accordance with the present invention can be assessed based on product quality analysis, reconstitution time (if lyophilized), quality of reconstitution (if lyophilized), high molecular weight, moisture, and glass transition temperature. Typically, protein quality and product analysis include product degradation rate analysis using methods including, but not limited to, size exclusion HPLC (SE-HPLC), cation exchange-HPLC (CEX-HPLC), X-ray diffraction (XRD), modulated differential scanning calorimetry (mDSC), reversed phase HPLC (RP-HPLC), multi-angle light scattering (MALS), fluorescence, ultraviolet absorption, nephelometry, capillary electrophoresis (CE), SDS-PAGE, and combinations thereof. In some embodiments, evaluation of product in accordance with the present invention may include a step of evaluating appearance (either liquid or cake appearance).

Generally, formulations (lyophilized or aqueous) can be stored for extended periods of time at room temperature. Storage temperature may typically range from 0° C. to 45° C. (e.g., 4° C., 20° C., 25° C., 45° C., etc.). Formulations may be stored for a period of months to a period of years. Storage time generally will be 24 months, 12 months, 6 months, 4.5 months, 3 months, 2 months or 1 month. Formulations can be stored directly in the container used for administration, eliminating transfer steps.

Formulations can be stored directly in the lyophilization container (if lyophilized), which may also function as the reconstitution vessel, eliminating transfer steps. Alternatively, lyophilized product formulations may be measured into smaller increments for storage. Storage should generally avoid circumstances that lead to degradation of the proteins, including but not limited to exposure to sunlight, UV radiation, other forms of electromagnetic radiation, excessive heat or cold, rapid thermal shock, and mechanical shock.

In some embodiments, formulations according to the present invention are in a liquid or aqueous form. In some embodiments, formulations of the present invention are lyophilized. Such lyophilized formulations may be reconstituted by adding one or more diluents thereto prior to administration to a patient. Suitable diluents include, but are not limited to, sterile water, bacteriostatic water for injection and sterile saline solution. Preferably, upon reconstitution, the antibody contained therein is stable, soluble and demonstrates tolerability upon administration to a patient.

The pharmaceutical compositions of the present invention are characterized by their tolerability. As used herein, the terms “tolerable” and “tolerability” refer to the ability of the pharmaceutical compositions of the present invention to not elicit an adverse reaction in the patient to whom such composition is administered, or alternatively not to elicit a serious adverse reaction in the patient to whom such composition is administered. In some embodiments, the pharmaceutical compositions of the present invention are well tolerated by the patient to whom such compositions is administered.

Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition that comprises the antibody disclosed herein may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Some embodiments include at least one of the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The formulations to be used for in vivo administration (e.g., subcutaneous administration) may be sterile. In some embodiments, the formulation is sterilized by filtration through sterile filtration membranes.

In some embodiments, the anti-CD19 antibodies (e.g., obexelimab) disclosed herein are formulated for subcutaneous (SC) administration. In some embodiments, the anti-CD19 antibody (e.g., obexelimab) formulation for SC administration comprises one or more buffers, one or more tonicity modifiers, one or more solvents, and one or more surfactants. Nonlimiting examples of buffers include phosphate, citrate, acetate, glutamate, carbonate, tartrate, triethanolamine (TRIS), glycylglycine, histidine, glycine, lysine, arginine, and other organic acids. More specifically, non-limiting examples of buffers include HEPES sodium, MES, potassium phosphate, potassium thiocyanate, sterilant, TAE, TBE, ammonium sulfate/HEPES, BuffAR, sodium acetate, sodium carbonate, sodium citrate, sodium dihydrogen phosphate, disodium hydrogen phosphate, and sodium phosphate. Additionally, the buffer may be various hydrate forms. For example, the buffer may be a monohydrate, a dihydrate, a trihydrate, a tetrahydate, a pentahydrate, a hexahydrate, a heptahydrate, an octahydrate, a nonahydrate, a decahydrate, an undecahydrate, and a dodecahydrate. Occasionally, a hydrate may be fractional such as a hemihydrate or a sequihydrate. Nonlimiting examples of tonicity modifies include sodium chloride, acetic acid, L-proline, dextrose, mannitol, potassium chloride, glycerin, and glycerol. Non-limiting example of solvents include water, propylene glycol, polyethylene glycols, ethanol, dimethyl sulfoxide, N-methyl-2-pyrrolidone, glycofurol, Solketal™, glycerol formal, acetone, tetrahydrofurfuryl alcohol, diglyme, dimethyl isosorbide, and ethyl lactate. Non-limiting examples of solvents include polysorbates (e.g. polysorbate-20, polysorbate-80), polyoxyethylene sorbitan monooleate (Tween 80), sorbitan monooleate polyoxyethylene sorbitan monolaurate (Tween 20), sorbitan trioleate (span 85), lecithin, and polyoxyethylene polyoxypropylene copolymers (Pluronics, Pluronic F-68).

The amounts of anti-CD19 antibody (e.g., obexelimab), buffer, tonicity modifier, solvent, and surfactants may vary. In some embodiments, the anti-CD19 antibody (e.g., obexelimab) is formulated at a concentration of 125 mg/mL obexelimab, 2.35 mg/mL sodium acetate trihydrate, 0.17 mg/mL acetic acid (at density 1.053 g/mL), 30 mg/mL L-proline, 0.1 mg/mL polysorbate 80, pH 5.5. In some embodiments, the anti-CD19 antibody (e.g., obexelimab) is formulated at a concentration of 80-200 mg/mL obexelimab, 1.5-3 mg/mL sodium acetate trihydrate, 0. 1-0.2 mg/mL acetic acid (at density 1.053 g/mL), 10-50 mg/mL L-proline, 0.05-0.2 mg/mL polysorbate 80, pH 5.0-6.0. In some embodiments, the anti-CD19 antibody (e.g., obexelimab) is formulated at a concentration of 122-127 mg/mL obexelimab, 2.0-2.5 mg/mL sodium acetate trihydrate, 0.15-0.19 mg/mL acetic acid (at density 1.053 g/mL), 25-35 mg/mL L-proline, 0.05-0.15 mg/mL polysorbate 80, pH 5.0-6.0.

In certain embodiments, a SC formulation comprises the anti-CD19 antibody (e.g., obexelimab), one or more buffers, one or more tonicity modifiers, one or more solvents, and one or more surfactants. In some embodiments, the buffer can be a sodium acetate buffer. For example, the buffer can be sodium acetate trihydrate. In an embodiment, the tonicity modifier can be acetic acid, L-proline, and combinations thereof. In another embodiment, the solvent is water.

In some embodiments, the surfactant is a polysorbate. In some embodiments, the polysorbate is polysorbate-80. In some embodiments, a SC formulation comprises the anti-CD19 antibody (e.g., obexelimab), sodium acetate trihydrate, acetic acid and L-proline, water, and polysorbate-80.

In some embodiments, the sub-cutaneous (SC) formulation comprises the anti-CD19 antibody (e.g., obexelimab) in an amount from about 1 mg to about 500 mg per mL or about 50 mg to about 250 mg per mL or about 100 mg to about 250 mg per mL, sodium acetate trihydrate in an amount from about 1 to about 10 mg per mL or about 1 to about 5 mg per mL or about 1 to about 2.5 mg per mL, acetic acid and L-proline in an amount from about 5 to about 50 mg per mL or about 10 to about 50 mg per mL or about 20 to about 40 mg per mL, water up to about 1 mL, and polysorbate-80 in an amount from about 0.01 mg to about 1 mg per mL or about 0.01 to about 0.5 mg/ml or about 0.05 to about 0.2 mg/ml. Specifically, a SC formulation comprises the anti-CD19 antibody (e.g., obexelimab) in an amount from about 100 mg to about 250 mg/ml, sodium acetate trihydrate in an amount from about 1 to about 2.5 mg/ml, acetic acid and L-proline in an amount from about 20 to about 40 mg/ml, water up to about 1 mg/ml, and polysorbate-80 in an amount from about 0.05 to about 0.2 mg per ml.

In some embodiments, the subcutaneous formulation comprises obexelimab, a buffer, and a tonicity modifier. In some embodiments, the subcutaneous formulation comprises 100 mg/mL to 250 mg/mL obexelimab, an acetate buffer, and proline. In some embodiments, the subcutaneous formulation comprises 125 mg/mL obexelimab, an acetate buffer, and proline. In some embodiments, the subcutaneous formulation comprises 100 mg/mL to 250 mg/mL obexelimab, 5 to 40 mM acetate buffer, and 1% to 5% (w/v) proline. In some embodiments, the subcutaneous formulation comprises 125 mg/mL obexelimab, 20 mM acetate buffer, and 3% (w/v) proline, at pH 5 to 6. In some embodiments, the subcutaneous formulation comprises 125 mg/mL obexelimab, 20 mM acetate buffer, and 3% (w/v) proline at pH 5.5.

In some embodiments, the subcutaneous formulation comprises obexelimab, a buffer, a tonicity modifier, and a surfactant.

In some embodiments, the subcutaneous formulation comprises 100 mg/mL to 250 mg/mL obexelimab, an acetate buffer, proline, and polysorbate 80.

In some embodiments, the subcutaneous formulation comprises 100 mg/mL to 250 mg/mL obexelimab, 5 to 40 mM acetate buffer, 1% to 5% (w/v) proline, and 0.002% to 0.02% (w/v) polysorbate 80.

In some embodiments, the subcutaneous formulation comprises 125 mg/mL obexelimab, 20 mM acetate buffer, 3% (w/v) proline, and 0.01% (w/v) polysorbate 80, at pH 5 to 6.

In some embodiments, the subcutaneous formulation comprises 125 mg/mL obexelimab, 20 mM acetate buffer, 3% (w/v) proline, and 0.01% (w/v) polysorbate 80 at pH of 5.5.

In some embodiments, a subcutaneous formulation has any one of the features described in Table 2 herein. For example, in some embodiments, a subcutaneous formulation comprises 125 mg/mL obexelimab, 2.35 mg/mL sodium acetate trihydrate, 0.17 mg/mL acetic acid (at density 1.053 g/mL), 30 mg/mL L-proline, and 0.1 mg/mL polysorbate 80. In some embodiments, the subcutaneous formulation has a pH of 5.5.

In some embodiments, a subcutaneous formulation has a unit dose strength(s)/dosage level(s) that is 125.0 (+10%) mg/mL of obexelimab.

The anti-CD19 antibodies (e.g., obexelimab) disclosed herein may also be formulated as immunoliposomes. A liposome is a small vesicle comprising various types of lipids, phospholipids and/or surfactant that is useful for delivery of an anti-CD19 antibody (e.g., obexelimab) to a mammal. Liposomes containing the anti-CD19 antibody (e.g., obexelimab) are prepared by methods known in the art. Liposomes with enhanced circulation time are disclosed in U.S. Pat. No. 5,013,556, incorporated entirely by reference. In some embodiments, the anti-CD19 antibody (e.g., obexelimab) is formulated in liposomes generated by the reverse phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

In some embodiments, the anti-CD19 antibody (e.g., obexelimab) is entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980, incorporated entirely by reference.

In some embodiments, the anti-CD19 antibody (e.g., obexelimab) Sustained-release preparations may be prepared. Suitable examples of sustained-release preparations include semipermeable matrices of solid hydrophobic polymer, which matrices are in the form of shaped articles, e.g., films, or microcapsules. Examples of sustained-release matrices include polyesters, hydrogels (for example poly(2-hydroxyethyl-methacrylate), or poly(vinylalcohol)), polylactides (U.S. Pat. No. 3,773,919, incorporated entirely by reference), copolymers of L-glutamic acid and gamma ethyl-L-glutamate, non-degradable ethylene-vinyl acetate, degradable lactic acid-glycolic acid copolymers such as the Lupron Depot® (which are injectable microspheres composed of lactic acid-glycolic acid copolymer and leuprolide acetate), poly-D-(−)-3-hydroxybutyric acid, and ProLease® (commercially available from Alkermes), which is a microsphere-based delivery system composed of the desired bioactive molecule incorporated into a matrix of poly-DL-lactide-co-glycolide (PLG).

### Containers for Injection

The present invention provides containers for injecting the pharmaceutical compositions and formulations of anti-CD19 antibodies (e.g., obexelimab). In one aspect, the container comprises a liquid pharmaceutical composition comprising obexelimab. Suitable containers include, without limitation, a syringe, an autoinjector, vial, infusion bottle, ampoule, carpoule, a syringe equipped with a needle protection system, and a carpoule within an injection pen.

In some embodiments, the container comprising the liquid pharmaceutical composition is a prefilled syringe, a vial, or an autoinjector. In some embodiments, the container is a prefilled syringe. In some embodiments the container is an autoinjector. In some embodiments, the autoinjector administers a 2.0 mL fixed injection dose volume. In some embodiments, the autoinjector administers a 2.0 mL fixed injection dose volume using a 27-gauge needle. In some embodiments, the autoinjector is suitable for once weekly administration of an effective amount of the obexelimab composition to a patient (e.g., any formulation described herein).

In some embodiments, the container is a prefilled syringe or autoinjector comprising a liquid pharmaceutical composition comprising:
a. 122-127 mg/mL obexelimab,
b. 2.0-2.5 mg/mL sodium acetate trihydrate, at a pH 5.0-6.0.
c. 0.15-0.19 mg/mL acetic acid (at density 1.053 g/mL),
d. 25-35 mg/mL L-proline,
e. 0.05-0.15 mg/mL polysorbate 80.

In some embodiments, the prefilled syringe or autoinjector comprises a liquid pharmaceutical composition comprising: the liquid pharmaceutical composition comprises about 125 mg/mL obexelimab, 2.35 mg/mL sodium acetate trihydrate, 0.17 mg/mL acetic acid, 30 mg/mL L-proline, 0.1 mg/mL polysorbate 80 at pH 5.5.

In some embodiments, the prefilled syringe or autoinjector comprises a liquid pharmaceutical composition having a unit dose strength of 125.0 (+10%) mg/mL of obexelimab.

In some embodiments, the prefilled syringe or autoinjector comprises a liquid pharmaceutical composition comprising: about 125 mg/mL obexelimab, 20 mM acetate buffer, 3% (w/v) proline, 0.01% (w/v) polysorbate 80, at pH 5.5.

In some embodiments, the prefilled syringe or autoinjector facilitates subcutaneous or intradermal delivery of the pharmaceutical composition. In some embodiments, the method of treating AIHA (e.g., wAIHA) described herein comprises, administering the formulation comprising obexelimab into the patient's bloodstream following a single or multiple subcutaneous injection to the abdomen of the patient using the prefilled syringe or autoinjector.

In some embodiments, the method of treating AIHA (e.g., wAIHA) comprises administering a single weekly injection of a 2.0 mL fixed injection dose. In some embodiments, the method of treating AIHA (e.g., wAIHA) comprises administering a single weekly injection of a 2.0 mL of 125 mg/mL obexelimab, 20 mM acetate buffer, 3% (w/v) proline, 0.01% (w/v) polysorbate 80, at pH 5.5.

In some embodiments, the composition in the prefilled syringe is stable for at least 3 months when stored at 2-8° C. In some embodiments, the composition in the pre-filled syringe is stable for at least 6 months when stored at 2-8° C. In some embodiments, the composition in the pre-filled syringe is stable for at least 6 months when stored at 2-8° C. In some embodiments, the composition in the pre-filled syringe is stable for at least 1 year when stored at 2-8° C. In some embodiments, the composition in the pre-filled syringe is stable for at least 2 years when stored at 2-8° C.

### Autoimmune Hemolytic Anemia (AIHA)

Provided herein are methods of treating AIHA comprising administering obexelimab. Hemolytic anemia occurs when RBCs undergo hemolysis more rapidly than restored. Severe anemia is considered as a Hgb<10.0 g/dL. Autoimmune hemolytic anaemias (AIHA) are acquired haematological disorders caused by increased peripheral erythrocyte destruction mediated by autoantibodies against erythrocyte (RBC) antigens. Autoimmune hemolytic anemia (AIHA) may occur with or without complement activation. In fact, critical to the diagnosis of AIHA is the identification of antibodies against RBC antigens. For example, RBCs are initially assessed for the presence of both immunoglobulin and C3 using polyspecific antisera. The etiology underlying the pathogenesis of such autoantibodies is still uncertain. Known associations with autoimmune hemolytic anemia (AIHA) include lymphoproliferative neoplasms, autoimmune conditions, drug use and viral infections. The clinical picture is heterogenous and may range from mild/compensated to life-threatening anemia, depending on the antibody's thermal amplitude, isotype and ability to fix complement, as well as on bone marrow compensation. Steroids, immunosuppressants and splenectomy have been the mainstay of treatment. One of the many clinical challenges for the treatment of AIHA is refractory to treatment or recurrence of AIHA. An atypical setting is AIHA after autologous and allogeneic hematopoietic stem cell transplantation. These cases are generally severe and refractory to standard therapy and have high mortality. AIHAs may be primary/idiopathic or secondary to infections, autoimmune diseases, malignancies, particularly lymphoproliferative disorders, and drugs, further complicating their clinical picture and management. Regarding new drugs, the false positivity of the Coombs test (direct antiglobulin test, DAT) following daratumumab adds to the list of difficult diagnosis, together with the passenger lymphocyte syndrome after solid organ transplants. AIHA is also increasingly described following therapy of solid cancers with inhibitors of immune checkpoint molecules.

Warm Autoimmune Hemolytic Anemia (wAIHA)

Warm reactive autoantibodies are encountered relatively frequently in tertiary care hospitals. wAIHA is characterized by evidence of RBC hemolysis and a DAT positive for IgG and sometimes complement. While varying with the extent of the compensatory increase in RBC production, symptoms of anemia predominate, as does jaundice, the latter often exacerbated by concurrent Gilbert's syndrome. Rarely, antibodies may be IgA and even less commonly, IgM; these may account for the approximately 5% of patients who have “Coombs-negative” wAIHA.

More than 95% of patients with wAIHA have an IgG antibody that binds to RBC antigens independently of temperature. The antibodies bind two major RBC antigens-Rh proteins and Glyphorins. Without wishing to be bound by theory, while autoantibodies binding to Rh proteins are not likely to activate complement, due to the number present on RBCs, autoantibodies binding to glyphorins may activate complement. Once bound to an antibody, the RBC can undergo multiple fates. Most will be bound to splenic macrophages via the FcγRIII receptor, resulting in either phagocytosis of the entire RBC or, removal of a significant portion of the RBC membrane-producing spherocytes (also called microspherocytes). Unlike the normal RBC, spherocytes are not deformable and, upon entering the splenic Cords of Billroth, undergo destruction. This may account for some of the increased LDH and depressed haptoglobin. About one-third of patients with wAIHA additionally have complement bound to RBCs, and these C3b-coated RBCs are then cleared by the C3b receptors on hepatic Kupffer cells. In some of these RBCs, C3b is inactivated to C3d, thereby preventing their subsequent destruction and allowing a population of antibody/complement-coated RBCs to persist.

In summary, wAIHA may be defined by the following: (1) evidence of hemolysis; (2) an IgG antibody binding to protein antigens on the RBC surface at the core temperature (warm antibody), demonstrated by a positive Coombs test for IgG in 95% of patients; (3) spherocytes on the peripheral blood smear.

Patients with wAIHA present with typical symptoms of anemia including shortness of breath with exercise, generalized fatigue, dizziness, syncope, malaise, chest pressure/pain, and cognitive dysfunction. Most patients have a reduced health-related quality of life. Some wAIHA patients have a mild anemia and increased mean corpuscular volume and are referred to hematology for assessment for possible myelodysplastic syndrome. The increased mean corpuscular volume is related to the increased numbers of reticulocytes (which are generally larger than more mature RBCs) and is not due to a primary bone marrow disorder.

In some embodiments, the patient has primary wAIHA. In some embodiments, the patient has secondary wAIHA. In some embodiments, the patient has secondary wAIHA due to an autoimmune disorder. In some embodiments the secondary wAIHA is due to systemic lupus erythematous, rheumatoid arthritis, deficiency of the immune system (immunodeficiency). In some embodiments, the patient has secondary wAIHA due to lymphoproliferative disorders, B-cell lymphoma or chronic lymphocytic leukemia (CLL). In some embodiments, the patient has secondary wAIHA due to infection or drug use such as methyldopa or carbamazepine.

Cold Autoimmune Hemolytic Anemia (cAIHA)

Cold-reactive autoimmune hemolytic anemia (cAIHA) or cold agglutinin disease (CAD) is rare among the hemolytic anemias. Cold AIHA is typically a primary lymphoproliferative disorder with marrow B-cell clones producing pathogenic IgM. More rarely, secondary cold AIHA (cAIHA) can develop from malignancy, infection, or other autoimmune disorders. Cold autoimmune hemolytic anemia (cAIHA) is caused by IgM autoantibodies whose K light chains bind erythrocyte I (or i) antigens at temperatures below 37° C. The pathogenic antibody is a cold agglutinin most often of the immunoglobulin M (IgM) subtype with I antigen specificity. Cold agglutinin fixes complement (Clq) below core body temperature and dissociates from the red cells as they move centrally, which leads to activation of the classical complement pathway and deposition of C3b on the RBC surface. Complement-coated erythrocytes are subject to extravascular hemolysis via C3b receptors in the hepatic mononuclear phagocytic system. If a large amount of complement is generated, formation of the membrane attack complex leads to intravascular hemolysis. This process is generally limited by the presence of complement regulatory proteins (CD55 and CD59) on the red blood cell surface.

In patients with cAIHA, DAT is positive for presence of C3d. Hemolysis with an elevated reticulocyte count and positive hemolytic markers is also observed. The mean corpuscular volume is elevated, reflecting reticulocytosis, or falsely elevated due to RBC agglutination. RBC agglutination is apparent on peripheral smear.

Cold AIHA is commonly primary but can be secondary to another disorder. In adults, CAD is a clonal disorder driven by low-grade B-cell proliferation in the absence of an overt malignancy. Proliferating B cells produce IgM, which drives hemolysis through complement activation. CAD is more common than cold agglutinin syndrome (CAS), which is defined as cold hemolytic anemia arising secondary to another disorder such as autoimmune disease, infection, or malignancy.

In one aspect, the present invention provides a method of treating cold AIHA (cAIHA) comprising administering obexelimab according to the methods disclosed herein. The terms Cold-reactive autoimmune hemolytic anemia (cAIHA) and cold agglutinin disease (CAD) are used interchangeably in this disclosure.

### Mixed AIHA

Mixed autoimmune hemolytic anemia (AIHA) is defined by the presence of both warm and cold auto antibodies. Diagnosed by detecting autoantibodies by monospecific direct antiglobulin test showing a pattern of IgG and complement C3d and presence of cold agglutinins, mixed AIHA accounts for less than 10% of all cases. Such variants of AIHA can be idiopathic or secondary to lymphoproliferative disorders or systemic lupus erythematosus (SLE). Patients with mixed AIHA often experience chronic course with periodic severe exacerbations. The hemoglobin can drop below 5 g/dL. The hematological screens show pan-agglutination with IgG. DAT is positive for IgG and C3d.

In one aspect, the present invention provides a method of treating mixed AIHA comprising administering obexelimab according to the methods disclosed herein.

### Paroxysmal Cold Hemoglobinuria (PCH)

Paroxysmal cold hemoglobinuria (PCH) is a rare form of cold autoimmune hemolytic anemia first discovered in the early 20th century in adults with tertiary syphilis. Initially described as a chronic relapsing condition in adults with tertiary syphilis, today, the acute form of PCH in children is most commonly described with a median age of five years. PCH comprises 30-40% of all pediatric cases of autoimmune hemolytic anemia. Certain viruses have been implicated in acute episodes of PCH in children, including measles, mumps, varicella, cytomegalovirus, EBV, influenza, parvovirus B19, coxsackievirus, and adenovirus.

The diagnosis of PCH is confirmed by the presence of the DL antibody, a biphasic, polyclonal IgG that binds to the surface of RBCs, most commonly the p-antigen, in the colder temperature of the extremities. The antibody fixes the first two components of the complement cascade and then dissociates upon rewarming, resulting in complement-mediated intravascular hemolysis. Due to this biphasic nature, DAT is negative for IgG and positive for anti-C3. In the differential diagnosis of PCH, cold agglutinin disease has the same DAT results but a negative DL test (named after Julius Donath and Karl Landsteiner who discovered this test). Furthermore, IgM will be positive, and the peripheral smear will show RBC agglutination.

A DL test can be performed by either directly using whole blood or indirectly using separated serum. In the direct test, two blood specimens are collected and immediately incubated at 37° C. The test specimen is cooled to 0° C. for one hour and then rewarmed to 37° C. for thirty minutes while the control specimen is maintained at 37° C. for the entire procedure. The supernatant serum of the chilled sample should demonstrate signs of hemolysis, indicating a positive test.

Given the self-resolving nature of acute PCH, management primarily involves avoidance of cold temperatures. Warming the patient, in addition to warming all ingestions or intravenous fluids, can prevent continued hemolysis. Depending on the severity of the anemia, transfusions can be given, ideally with warmed blood lacking the p-antigen. For chronic PCH, cold avoidance remains the mainstay of treatment. Glucocorticoids have historically been used for refractory anemia but there is insufficient evidence to demonstrate any benefit.

In one aspect, the present invention provides a method of treating PCH comprising administering obexelimab according to the methods disclosed herein.

### Human Patient Population

The present disclosure provides, methods of selecting patients for treatment with obexelimab.

In some embodiments, patients are selected based on confirmed diagnosis of wAIHA.

In some embodiments, patients who have a Hgb level of ≥7 g/dL are selected.

In some embodiments, patients with a Hgb level of at or greater than 7 g/dL are selected. In some embodiments, patients with a Hgb level of at or greater than 8 g/dL are selected. In some embodiments, patients with a Hgb level of at or greater than 9 g/dL are selected. In some embodiments, patients with a Hgb level of at or greater than 10 g/dL are selected.

In some embodiments, eligible human patients have an Hgb level of 11 g/dL or less than 11 g/dL. In some embodiments, eligible human patients have an Hgb level of 10 g/dL or less than 10 g/dL. In some embodiments, eligible human patients have an Hgb level of 9 g/dL or less than 9 g/dL. In some embodiments, eligible human patients have an Hgb level of 8 g/dL or less than 8 g/dL. In some embodiments, eligible human patients have an Hgb level of 7 g/dL or less than 7 g/dL.

Eligible human patients according to the study are patients with a diagnosis of wAIHA with a Hgb level of 10 g/dL or less than 10 g/dL. In some embodiments, patients selected for treatment have at least one sign or symptom of anemia, which include, but are not limited to fatigue, weakness, pallor, chest pain or shortness of breath (at rest or with exertion), headache, dizziness, or lightheadedness, tachycardia or tachypnea.

In some embodiments, patients also have failed at least 1 prior wAIHA treatment regimen (GC or immunosuppression therapy). In some embodiments, a patient is considered to have failed therapy if, during the course of the disease, there was a reduction in Hgb of 1 g/dL or more and an increase in LDH of ≥1.5× upper limit of normal (ULN). In some embodiments, failure is assessed after a minimum of 4 weeks of GC therapy. In some embodiments, failure is assessed at after a minimum of 3 months of immunosuppression therapy.

In some embodiments, patients have failed up to 2 prior wAIHA therapies. In some embodiments, a prior failed therapy may be considered concurrent therapy, if the patient maintains a Hgb level of >7 g/dL. In some embodiments, a prior failed therapy may be considered concurrent therapy, if the patient maintains a Hgb level of ≥8 g/dL. In some embodiments, a prior failed therapy may be considered concurrent therapy, if the patient maintains a Hgb level of >9 g/dL. In some embodiments, a prior failed therapy may be considered concurrent therapy, if the patient maintains a Hgb level of <10 g/dL on the therapy.

In some embodiments, patients receiving GCs must be on a stable dose, not to exceed 20 mg/day of prednisone or prednisolone, for 4 weeks prior to the start of treatment with obexelimab.

In some embodiments, patients receiving immunosuppressants are on a stable dose for 12 weeks and remain on a stable dose of immunosuppressants. Exemplary immunosuppressants include azathioprine, mycophenolate mofetil/mycophenolic acid, cyclosporine, and cyclophosphamide.

In some embodiments, the prior therapy is GC therapy and/or an increase in background long-term GC therapy.

In some embodiments, eligible patients have received at least 2 weeks of GC treatment. In some embodiments, eligible patients according to the invention, are required to receive at least 3 weeks of GC treatment. In some embodiments, eligible patients according to the invention, are required to receive more than 3 weeks of GC treatment. In some embodiments, eligible patients according to the invention, are required to receive up to a maximum of 6 weeks of GC treatment. In some embodiments, the GC treatment is administered at a dose of 30-50 mg/day prednisone or equivalent. In some embodiments, the GC treatment is administered at a dose of 10-70 mg/day prednisone or equivalent. In some embodiments, the GC treatment is administered at a dose of 20-60 mg/day prednisone or equivalent.

In some embodiments, in eligible patients according to the invention, the GC therapy is administered along with obexelimab. In some embodiments, the GC treatment is tapered before the start of obexelimab therapy. Exemplary taper protocols involve administration of corticosteroids at 0.6-1.0 mg/kg daily for 2-4 weeks followed by a gradual taper. In some exemplary embodiments, GC is completely tapered within 8-12 weeks, to discontinuation. In some embodiments the tapering takes less than 8 weeks. In some embodiments, the tapering takes more than 12 weeks. In some exemplary embodiments, GC treatment is allowed to continue at a low to moderate dose of 2.5-10.0 mg daily for up to several years.

In some embodiments, eligible patients according to the invention, can have plasmablast levels greater than 100 cells/mL, greater than 200 cells/mL, greater than 300 cells/mL, greater than 400 cells/mL, greater than 500 cells/mL, greater than 600 cells/mL, greater than 700 cells/mL, greater than 800 cells/mL, greater than 900 cells/mL, greater than 1000 cells/mL, greater than 2000 cells/mL, greater than 3000 cells/mL, greater than 4000 cells/mL or greater than 5000 cells/mL.

In some embodiments, obexelimab is administered every 7 days. In some embodiments, obexelimab is administered at the study site for the first 5 weeks and then every 2 weeks (Q2W) thereafter during required in-clinic visits (Day 1 [Week 0] and Weeks 1, 2, 3, 4, 6, 8, 10, 12, etc.). In some embodiments, all patients will be observed for at least 2 hours following the first SC administration of obexelimab, during which time safety assessments will be performed; for subsequent SC administrations, patients may be discharged from the clinic following obexelimab administration when deemed safe and will be instructed on at-home obexelimab administration. In some embodiments, blood draw for collection of PK, PD, and anti-drug antibody (ADA) sampling are conducted 24 and 72 hours post Day 1.

Treatment of AIHA with Obexelimab

Provided herein are methods of treating AIHA (e.g., wAIHA) by administering obexelimab. In some embodiments, treatment with obexelimab generates a durable Hgb response without the need for long-term steroid use that is associated with substantial toxicity and complications. Obexelimab (XmAb5871) is a humanized anti-CD19 monoclonal antibody with an Fc portion engineered for increased affinity to FcγRIIb, the only receptor on B-cells.

In some embodiments, administration of obexelimab in a subcutaneous (SC) formulation minimizes the risk of acute administration-related reactions.

In some embodiments, a patient is an adult human patient (e.g., a human patient≥ 18 years of age).

In some embodiments, a patient has been diagnosed with wAIHA for at least three (3) months.

In some embodiments, a patient is receiving treatment for wAIHA at the time treatment comprising administering obexelimab commences.

In some embodiments, a patient is not receiving treatment for wAIHA at the time treatment comprising administering obexelimab commences.

In some embodiments, a patient has previously received treatment for wAIHA prior to treatment comprising administering obexelimab commences.

In some embodiments, a patient is diagnosed with primary or secondary wAIHA (e.g., as documented by a positive DAT specific for anti-IgG or anti-IgA). In some embodiments, a patient is diagnosed with primary wAIHA. In some embodiments, a patient is diagnosed with secondary wAIHA.

In some embodiments, a patient received at least one (1) prior wAIHA treatment regimen (e.g., one or more treatment regimens comprising the administration of steroids, rituximab, azathioprine, cyclophosphamide, cyclosporine, mycophenolate mofetil, danazol, vincristine, or erythropoiesis-stimulating agents, or wherein the patient previously underwent splenectomy, or any combination thereof).

In some embodiments, a patient is receiving a steroid (e.g., prednisone/prednisolone) concomitantly with treatment comprising administration of obexelimab as described herein. In some embodiments, a dose of a steroid (e.g., prednisone/prednisolone) may not exceed 20 mg/day, is stable for at least 4 weeks prior to randomization, and/or remains stable during treatment (for example, throughout the dose confirmation run-in period (SRP) and/or randomized-control period (RCP)).

In some embodiments, a patient is receiving an immunosuppressant concomitantly with treatment comprising administration of obexelimab as described herein. In embodiments, an immunosuppressant is selected from the group consisting of azathioprine, mycophenolate mofetil/mycophenolic acid, cyclosporine, and cyclophosphamide, and any combinations thereof. In some embodiments, a patient is on a stable dose during treatment (for example, for at least 12 weeks prior to randomization and/or throughout the SRP and/or RCP).

In some embodiments, a patient has Hgb≥7 to <10 g/dL at the time treatment comprising administration of obexelimab as described herein is commenced.

In some embodiments, a patient has least one sign or symptom of anemia at the time treatment comprising administration of obexelimab as described herein is commenced.

In some embodiments, a patient has a platelet count≥50,000 mm³ at the time treatment comprising administration of obexelimab as described herein is commenced.

In some embodiments, a patient has a neutrophil count≥1,000 mm³ at the time treatment comprising administration of obexelimab as described herein is commenced.

In some embodiments, a patient has a serum albumin and serum calcium concentrations within the normal range at the time treatment comprising administration of obexelimab as described herein is commenced.

In some embodiments, a patient has a total serum IgG of >600 mg/dL at the time treatment comprising administration of obexelimab as described herein is commenced.

In some embodiments, a patient has a creatine kinase value<2×ULN at the time treatment comprising administration of obexelimab as described herein is commenced.

In some embodiments, a patient who has previously undergone splenectomy is at least 4 months post resection at the time treatment comprising administration of obexelimab as described herein is commenced. In embodiments, the patient is vaccinated as per country-specific immunization schedules

In some embodiments, a patient has an autoimmune disorder. In embodiments, an autoimmune disorder is systemic lupus erythematosus or rheumatoid arthritis. In embodiments, an autoimmune disorder is a lymphoproliferative disorder (LPD). In embodiments, a patient with an autoimmune disorder has stable disease with respect to the autoimmune disorder (e.g., a patient has no changes in disease-related concomitant medications and/or the severity of disease has been stable (e.g., for at least 4 months)).

In some embodiments, a patient has a lymphoproliferative disorder (LPD). In embodiments, a patient with an LPD has stable disease with respect to the LPD (e.g., a patient has no changes in concomitant disease-related medications and/or the severity of disease has been stable (e.g., for at least 4 months)).

In some embodiments, a patient does not have cold antibody AIHA, cold agglutinin syndrome, mixed type (i.e., warm and cold) AIHA, or paroxysmal cold hemoglobinuria.

In some embodiments, a patient does not have any other associated cause of hereditary or acquired hemolytic anemia.

In some embodiments, a patient does not have secondary wAIHA that is not due to an autoimmune disorder (e.g., a LPD).

In some embodiments, a patient has not received a transfusion within 2 weeks prior to commencing treatment comprising administration of obexelimab as described herein.

In some embodiments, a patient has not received B cell-depleting, B cell-targeted, or other biologic immunomodulatory agents within the 6 months prior to commencing treatment comprising administration of obexelimab as described herein.

In some embodiments, a patient has received B cell-depleting, B cell-targeted, or other biologic immunomodulatory agents within 6-12 months prior to commencing treatment comprising administration of obexelimab as described herein. In some embodiments, the patient has a B cell count that is within the laboratory reference range at the time treatment with obexelimab is commenced.

In some embodiments, a patient has not received IV Ig or epoetin alfa within 6 weeks prior to commencing treatment comprising administration of obexelimab as described herein.

In some embodiments, a patient is not receiving more than two (2) concomitant medications for the treatment of wAIHA at the time treatment comprising administration of obexelimab as described herein is commenced. In some embodiments, the concomitant medications exclude vitamins or other supplements.

In some embodiments, a patient has not received an investigational treatment or direct medical intervention in another clinical study within 12 weeks or <5 half-lives of the investigational treatment (whichever is shorter) at the time treatment comprising administration of obexelimab as described herein is commenced.

In some embodiments, a patient has not received live vaccine or live therapeutic infectious agent within the 6 weeks prior to commencing treatment comprising administration of obexelimab as described herein.

In some embodiments, a patient does not evidence active tuberculosis (TB) or at high risk for TB. In some embodiments, criteria used to assess the patient are selected from:
History of active TB or latent TB, unless completion of treatment is documented;
Positive, indeterminate, or invalid interferon-gamma (IFNγ) release assay results at screening, unless treatment is documented (patients with an indeterminate test result can repeat the test once);
Signs of symptoms that could represent active TB; and/or
Chest radiograph, computed tomography scan, or magnetic resonance imaging that suggests possible diagnosis of TB.

In some embodiments, a patient does not have a history or evidence of a clinically unstable/uncontrolled disorder, condition, or disease (including, but not limited to, cardiopulmonary, oncologic, renal, hepatic, metabolic, hematologic, psychiatric, or active infection).

In some embodiments, a patient does not have a known allergy to monoclonal antibody therapy.

In some embodiments, a patient does not have a known hypersensitivity to dextran or components of dextran.

In some embodiments, a patient does not have an active infection (e.g., pneumonia, biliary tract infection, diverticulitis, Clostridium difficile infection) that requires parenteral or oral anti-infectives and/or hospitalization, and/or is assessed as serious/clinically significant within eight (8) weeks prior to commencing treatment comprising administration of obexelimab as described herein.

In some embodiments, a patient does not have a chronic infection (e.g., bronchiectasis, chronic osteomyelitis, chronic pyelonephritis) or requiring chronic treatment with anti-infectives (e.g., antibiotics, antivirals) prior to commencing treatment comprising administration of obexelimab as described herein.

In some embodiments, a patient does not have confirmed or suspected clinical immunodeficiency syndrome not related to treatment of wAIHA, or has a family history of congenital or hereditary immunodeficiency, unless confirmed absent in the patient prior to commencing treatment comprising administration of obexelimab as described herein.

In some embodiments, a patient does not have an acute hepatitis B infection (hepatitis B surface antigen-positive), active hepatitis C virus (HCV), or HIV infection prior to commencing treatment comprising administration of obexelimab as described herein.

In some embodiments, a patient does not have a positive test for active hepatitis B through detection of hepatitis B surface antigen prior to commencing treatment comprising administration of obexelimab as described herein.

In some embodiments, a patient does not have any of the following prior to commencing treatment comprising administration of obexelimab as described herein:
hepatitis B surface antigen,
hepatitis B surface antibody, or
hepatitis B core antibody.

In some embodiments, a patient does not have a history of HCV prior to commencing treatment comprising administration of obexelimab as described herein. In embodiments, a patient has documented negative HCV ribonucleic acid levels in the serum at 12 weeks or longer after the completion of HCV therapy prior to commencing treatment comprising administration of obexelimab as described herein.

Table 2 shows an exemplary dosage regimen and formulation of Obexelimab.

TABLE 2
Obexelimab Pre-filled syringe and dosage

| | |
|---|---|
| Study Investigational Product: | obexelimab |
| Dosage formulation: | Solution for injection |
| Route of administration: | Subcutaneous |
| Formulation: | Formulation: 125 mg/mL obexelimab, |
| | 2.35 mg/mL sodium acetate trihydrate, |
| | 0.17 mg/mL acetic acid |
| | (at density 1.053 g/mL), |
| | 30 mg/mL L-proline, |
| | 0.1 mg/mL polysorbate 80, pH 5.5 |
| Unit dose strength(s)/ | 125.0 (±10%) mg/mL |
| dosage level(s): | |
| Dosing instructions: | 2 × 1.0 mL administered SC |
| | to the abdominal region |
| Packaging: | Single-use 2-mL glass vials, |
| | each filled with 1.2 mL of drug |
| | product (e.g., a pre-filled syringe) |

In some embodiments, obexelimab is administered at 250 mg per week subcutaneously, at a concentration of 125 mg/ml, as 2×1 ml injections. In some embodiments, obexelimab is administered at 250 mg per week subcutaneously, at a concentration of 125 mg/ml, as 1×2 ml injections. In some embodiments, obexelimab is administered at 200 mg per week subcutaneously. In some embodiments, obexelimab is administered at 150 mg per week subcutaneously. In some embodiments, obexelimab is administered at 100 mg per week subcutaneously. In some embodiments, obexelimab is administered as a single injection. In some embodiments, obexelimab is administered as more than 2 injections, for example, obexelimab is administered as 3 injections, or 4 injections or more. In some embodiments, the concentration of obexelimab is 100 mg/ml. In some embodiments, the concentration of obexelimab is 50 mg/ml. In some embodiments, obexelimab is administered at a concentration of 100 mg/ml, as 2×1 ml injections, subcutaneously. In some embodiments, obexelimab is administered at a concentration of 125 mg/ml, as 2×1 ml injections, subcutaneously.

In one aspect, the present invention provides a method of treating wAIHA, comprising administering obexelimab subcutaneously to a human patient 18 years of age or older at a dose of 250 mg per week.

In some embodiments, the method comprises administering obexelimab subcutaneously at a dose of 125 mg twice a week. In some embodiments, the method comprises administering obexelimab subcutaneously at a dose of 125 mg twice a week. In some embodiments, the method comprises administering obexelimab subcutaneously at a dose of 125 mg every 3 days. In some embodiments, the method comprises administering obexelimab subcutaneously at a dose of 250 mg every 7 days.

In some embodiments, obexelimab can be administered subcutaneously to a human patient 18 years of age or older at a dose of 200 mg once a week. In some embodiments, obexelimab can be administered subcutaneously to a human patient 18 years of age or older at a dose of 125 mg twice a week. In some embodiments, obexelimab is administered subcutaneously to a human patient 18 years of age or older at a dose of 100 mg twice a week. In some embodiments, obexelimab is administered subcutaneously to a human patient 18 years of age or older at a dose of 300 mg once a week. In some embodiments, obexelimab is administered subcutaneously to a human patient 18 years of age or older at a dose of 150 mg twice a week.

In some embodiments, obexelimab is administered in a liquid formulation comprising 125 mg/mL obexelimab. In some embodiments, obexelimab is administered as 2×1 mL injections. In some embodiments, obexelimab is administered as 2×1 mL injections for a total dose of 250 mg. In some embodiments, obexelimab injections are administered concurrently. In some embodiments, obexelimab injections are administered within 1 minute, 2 minutes, 3 minutes, 4 minutes, 5 minutes, 6 minutes, 7 minutes, 8 minutes, 9 minutes, 10 minutes, or within 20 minutes of each other. In some embodiments, obexelimab injections are administered within 1 hour, within 2 hours, within 3 hours, within 4 hours, within 5 hours, within 6 hours, within 7 hours, within 8 hours, within 9 hours, within 10 hours, within 11 hours, or within 12 hours of each other.

In some embodiments, obexelimab is administered in a liquid formulation comprising 125 mg/mL obexelimab as 4×0.5 mL injections. In some embodiments, obexelimab is administered in a liquid formulation comprising 125 mg/mL obexelimab as 4×0.5 mL injections for a total dose of 250 mg. In some embodiments, obexelimab injections are administered concurrently. In some embodiments, obexelimab injections are administered within 1 minute, 2 minutes, 3 minutes, 4 minutes, 5 minutes, 6 minutes, 7 minutes, 8 minutes, 9 minutes, 10 minutes, or within 20 minutes of each other. In some embodiments, obexelimab injections are administered within 1 hour, within 2 hours, within 3 hours, within 4 hours, within 5 hours, within 6 hours, within 7 hours, within 8 hours, within 9 hours, within 10 hours, within 11 hours, or within 12 hours of each other.

In some embodiments, obexelimab is administered in a liquid formulation as a single injection. In some embodiments, obexelimab is administered in a liquid formulation as a single injection for a total dose of 250 mg.

In some embodiments, obexelimab is administered at a dose of about 100-500 mg per week. In some embodiments, obexelimab is administered at a dose of about 100-200 mg per week, 100-300 mg per week, 100-400 mg per week, 200-400 mg per week, 100-300 mg per week, 200-350 mg per week, 150-300 mg per week, 125-275 mg per week, 225-275 mg per week, 230-260 mg per week, 240-260 mg per week, or 245-255 mg per week. In some embodiments, obexelimab is administered at a dose of about 100 mg per week, 150 mg per week, 200 mg per week, 250 mg per week, 300 mg per week, 350 mg per week, 400 mg per week, 450 mg per week, or 500 mg per week.

In some embodiments, the weekly dose may be injected at once or divided into two or more injections throughout a week. In some embodiments, the weekly dose is divided into 2 injections, 3 injections, 4 injections, 5 injections, 6 injections or daily injections. In some embodiments, the weekly dose is divided equally. In some embodiments, the weekly dose is divided unequally. In some embodiments, the weekly dose is divided such that it is administered every other day, every 2 days or every 3 days.

In some embodiments, a suitable dose ranges from 20-40 mg daily, 25-40 mg daily, or 30-40 mg daily. In some embodiments, a suitable dose is 35 mg daily.

In some embodiments, obexelimab is administered at a dose of 500 mg every two weeks. In some embodiments, every 2-week dose may be injected at once or divided into two or more injections. In some embodiments, the dose is divided into 2 injections, 3 injections, 4 injections, 5 injections, 6 injections or more injections.

In some embodiments, the patient receives 1000 mg of obexelimab per month. In some embodiments, the 1000 mg monthly dose may be injected at once or divided into 4 or more injections. In some embodiments, the dose is divided into 4 injections, 5 injections, 6 injections, 7 injections, 8 injections or more injections.

In some embodiments, obexelimab is administered to a patient (e.g., once weekly subcutaneous administration of obexelimab) for a period of at least about 1-24, 4-24, 8-24, 12-24, 16-24, or 20-24 weeks. In some embodiments, obexelimab is administered to a patient (e.g., once weekly subcutaneous administration of obexelimab) for a period of about 1-24, 4-24, 8-24, 12-24, 16-24, or 20-24 weeks.

In some embodiments, obexelimab is administered to a patient (e.g., once weekly subcutaneous administration of obexelimab) for a period of at least about 4 weeks. In some embodiments, obexelimab is administered to a patient (e.g., once weekly subcutaneous administration of obexelimab) for a period of about 4 weeks. In some embodiments, obexelimab is administered to a patient (e.g., once weekly subcutaneous administration of obexelimab) for a period of at least about 8 weeks. In some embodiments, obexelimab is administered to a patient (e.g., once weekly subcutaneous administration of obexelimab) for a period of about 8 weeks. In some embodiments, obexelimab is administered to a patient (e.g., once weekly subcutaneous administration of obexelimab) for a period of at least about 12 weeks. In some embodiments, obexelimab is administered to a patient (e.g., once weekly subcutaneous administration of obexelimab) for a period of about 12 weeks. In some embodiments, obexelimab is administered to a patient (e.g., once weekly subcutaneous administration of obexelimab) for a period of at least about 16 weeks. In some embodiments, obexelimab is administered to a patient (e.g., once weekly subcutaneous administration of obexelimab) for a period of about 16 weeks. In some embodiments, obexelimab is administered to a patient (e.g., once weekly subcutaneous administration of obexelimab) for a period of at least about 20 weeks. In some embodiments, obexelimab is administered to a patient (e.g., once weekly subcutaneous administration of obexelimab) for a period of about 20 weeks. In some embodiments, obexelimab is administered to a patient (e.g., once weekly subcutaneous administration of obexelimab) for a period of at least about 24 weeks. In some embodiments, obexelimab is administered to a patient (e.g., once weekly subcutaneous administration of obexelimab) for a period of about 24 weeks.

Table 3 illustrates exemplary benefits that can be obtained using methods described herein. In some embodiments, methods described herein can attain one or more of the benefits described in Table 3.

TABLE 3
Exemplary benefits of methods of treatment
Endpoint

Change from Baseline to Week 24 in Hgb concentration
Proportion of patients who attain normal values on
at least 3 of 4 consecutive available visits, at the
earliest on or after Week 12, with no use of blood
transfusion or GC rescue therapy prior to attaining
durable response in all 3 of the following:
LDH
Haptoglobin
Indirect bilirubin
Proportion of patients with a ≥3-point increase in
FACIT-F score at Week 24 with no prior use of blood
transfusion or GC rescue therapy
Time to durable Hgb response in patients who
achieve the primary endpoint
Percentage of time in Hgb response, on or after Week 4
through Week 24, in patients who achieve the primary endpoint

In some embodiments, methods described herein result in a durable Hgb response (e.g., Hgb≥10 g/dL and/or ≥2 g/dL increase from baseline) in a patient. In embodiments, a durable response is obtained without use of blood transfusion or GC rescue therapy prior to attaining durable response.

In some embodiments, methods described herein result in a change in in FACIT-F score from Baseline to Week 24 of treatment according to methods described herein.

In some embodiments, methods described herein result in a beneficial change in the proportion of patients who do not receive blood transfusion or GC rescue therapy through week 24 of treatment according to methods described herein.

In some embodiments, methods described herein result in a beneficial change in the cumulative dose of GC rescue therapy through week 24 of treatment according to methods described herein.

In some embodiments, methods described herein result in a beneficial change in the proportion of patients who do not receive blood transfusions through week 24 of treatment according to methods described herein.

In some embodiments, methods described herein result in change from Baseline to week 24 in FACIT-F Score, EQ-5D-5L index score, Physician's Global Assessment of Change Disease Activity, Patient's Global Impression of Change in Daily Activity Impact, and/or Patient's Global Impression of Change in Fatigue Severity.

In some embodiments, methods described herein result in change from baseline through week 24 over time in one or more of the following:
Reduction in Circulating absolute T, B, and NK cell count;
Reduction in Ig levels and ratios (e.g., IgG, IgM, IgA, IgE);
Increase in CD19 target receptor occupancy;
Decrease in reticulocyte count;
Decrease in LDH;
Increase in Haptoglobin; and
Reduction in indirect bilirubin (unconjugated bilirubin).

In some embodiments, methods described herein result in significantly improved HgB levels in a patient (e.g., as compared to baseline HgB levels). In embodiments, an improvement is in Hgb concentration is observed by week 8 of treatment according to methods described herein. In embodiments, an improvement is in Hgb concentration is observed by week 24 of treatment according to methods described herein. In some embodiments, methods described herein result in a durable Hgb response (e.g., Hgb≥10 g/dL and/or ≥2 g/dL increase from baseline) in a patient. In embodiments, a durable response is obtained without use of blood transfusion or GC rescue therapy prior to attaining durable response.

In some embodiments, methods described herein result in a patient attaining normal values in lactate dehydrogenase (LDH), haptoglobin, and/or indirect bilirubin. In some embodiments, methods described herein result in a patient attaining normal values in lactate dehydrogenase (LDH), haptoglobin, and indirect bilirubin. In embodiments, the normal values for LDH, haptoglobin, and/or indirect bilirubin are attained on or after Week 12 of treatment according to methods described herein. In embodiments, the normal values for LDH, haptoglobin, and/or indirect bilirubin are attained without use of blood transfusion or GC rescue therapy.

In some embodiments, methods described herein result in a patient attaining≥3-point increase in FACIT-F score. In embodiments, a patient attains≥3-point increase in FACIT-F score on or after Week 24 of treatment according to methods described herein. Ine embodiments, the increase in FACIT-F score is obtained with no prior use of blood transfusion or GC rescue therapy.

### Durable Hgb Response:

A durable Hgb response can be Hgb≥10 g/dL or ≥2 g/dL increase from baseline. In some embodiments, durable Hgb response is achieved after at least 3 of 4 consecutive weeks. In some embodiments, the Hgb values increase at the earliest in less than one week. In some embodiments, the Hgb values increase at the earliest on or after one week. In some embodiments, the Hgb values increase at the earliest on or after 2 weeks. In some embodiments, the Hgb values increase at the earliest on or after 3 weeks. In some embodiments, the Hgb values increase at the earliest on or after 4 weeks. In some embodiments, the Hgb values increase at the earliest on or after 5 weeks. In some embodiments, the Hgb values increase at the earliest on or after 6 weeks. In some embodiments, the Hgb values increase at the earliest on or after 7 weeks. In some embodiments, the Hgb values increase at the earliest on or after 8 weeks. In some embodiments, the Hgb values increase at the earliest on or after 9 weeks. In some embodiments, the Hgb values increase at the earliest on or after 10 weeks. In some embodiments, the Hgb values increase at the earliest on or after 11 weeks. In some embodiments, the Hgb values increase at the earliest on or after 12 weeks. In some embodiments, patients achieving increase in Hgb did not use of blood transfusion or GC rescue therapy prior to attaining Hgb increase.

In some embodiments, a patient achieves≥0.5 g/dL increase from Baseline on at least 3 of 4 consecutive available visits. In some embodiments, a patient achieves≥1 g/dL increase from Baseline on at least 3 of 4 consecutive available visits. In some embodiments, a patient achieves≥1.5 g/dL increase from Baseline on at least 3 of 4 consecutive available visits. In some embodiments, a patient achieves≥2 g/dL increase from Baseline on at least 3 of 4 consecutive available visits.

In some embodiments, the increase in Hgb levels is sustained through week 13. In some embodiments, the increase in Hgb levels is sustained through week 14. In some embodiments, the increase in Hgb levels is sustained through week 15. In some embodiments, the increase in Hgb levels is sustained through week 16. In some embodiments, the increase in Hgb levels is sustained through week 17. In some embodiments, the increase in Hgb levels is sustained through week 18. In some embodiments, the increase in Hgb levels is sustained through week 19. In some embodiments, the increase in Hgb levels is sustained through week 20. In some embodiments, the increase in Hgb levels is sustained through week 21. In some embodiments, the increase in Hgb levels is sustained through week 22. In some embodiments, the increase in Hgb levels is sustained through week 23. In some embodiments, the increase in Hgb levels is sustained through week 24.

In some embodiments, Hgb response is defined as patients who achieve Hgb≥9 g/dL. In some embodiments, Hgb response is defined as patients who achieve Hgb≥10 g/dL. In some embodiments, Hgb response is defined as patients who achieve Hgb≥11 g/dL. In some embodiments, Hgb response is defined as patients who achieve Hgb≥12 g/dL. In some embodiments, Hgb response is defined as patients who achieve Hgb≥13 g/dL. In some embodiments, Hgb response is defined as patients who achieve Hgb≥14 g/dL. In some embodiments, Hgb response is defined as patients who achieve Hgb≥15 g/dL. In some embodiments, Hgb response is defined as patients who achieve Hgb≥16 g/dL. In some embodiments, Hgb response is defined as patients who achieve Hgb≥17 g/dL.

In some embodiments, a durable Hgb response is defined as patients who achieve≥2 g/dL increase from baseline. In some embodiments, patients may achieve≥0.5 g/dL increase from baseline. In some embodiments, patients may achieve≥1 g/dL increase from baseline. In some embodiments, patients may achieve≥3 g/dL increase from baseline. In some embodiments, patients may achieve≥4 g/dL increase from baseline. In some embodiments, patients may achieve≥5 g/dL increase from baseline. In some embodiments, patients may achieve>6 g/dL increase from baseline. In some embodiments, patients may achieve≥7 g/dL increase from baseline. In some embodiments, patients may achieve≥8 g/dL increase from baseline.

In some embodiments, a durable Hgb response maintains Hgb values for at 1 week, at least two weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks at least 14 weeks at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, or at least 25 weeks.

In some embodiments, the patient achieves an increase in Hgb levels in less than 1 week. some embodiments, the patient achieves an increase in Hgb levels in 2 weeks or less. some embodiments, the patient achieves an increase in Hgb levels in 3 weeks or less. some embodiments, the patient achieves an increase in Hgb levels in 4 weeks or less. some embodiments, the patient achieves an increase in Hgb levels in 5 weeks or less. some embodiments, the patient achieves an increase in Hgb levels in 6 weeks or less. some embodiments, the patient achieves an increase in Hgb levels in 7 weeks or less. some embodiments, the patient achieves an increase in Hgb levels in 8 weeks or less. some embodiments, the patient achieves an increase in Hgb levels in 9 weeks or less. some embodiments, the patient achieves an increase in Hgb levels in 10 weeks or less. some embodiments, the patient achieves an increase in Hgb levels in 11 weeks or less. some embodiments, the patient achieves an increase in Hgb levels in 12 weeks or less.

In some embodiments, the patient maintains Hgb values for at least 3 weeks with no use of blood transfusion or GC rescue therapy prior to attaining durable response.

### Quality of Life Assessment

Quality of life assessments may be performed as part of the treatment methods described herein. In some embodiments, enhanced quality of life is an improvement in one or more characteristics of fatigue or tiredness and its impact on daily activities and functioning. The assessment instrument may include items such as tiredness, weakness, listlessness, lack of energy, and the impact of these feelings on daily functioning (e.g., sleeping, social activities). In some embodiments, the present disclosure provides a method of treating autoimmune hemolytic anemia (AIHA), measuring the patients FACIT-F Score or EQ-5D-5L index score and administering obexelimab subcutaneously to a human patient at a dose of 250 mg once a week.

In some embodiments, the quality of life is measured by FACIT-F scale. In some embodiments, the Quality of life is measured by EQ-5D-5L Questionnaire. In some embodiments, the Quality of life is measured by SF-36 survey.

### Rescue Therapy

The present invention also provides methods of treating AIHA (e.g., wAIHA) comprising administering a rescue therapy. In some embodiments, the rescue therapy comprises administration of GC therapy or blood transfusion. In some embodiments, the rescue therapy is any therapy given for the treatment of wAIHA, due to a reduction in Hgb compared to previous Hgb level and/or an increase in LDH, with new or worsening anemia.

In some embodiments, the GC therapy is administered at a dose of 1-1.5 mg/kg. In some embodiments, the GC therapy is administered at a dose of 0.5-2 mg/kg. In some embodiments, the GC therapy is administered at a dose of 0.6 mg/kg. In some embodiments, the GC therapy is administered at a dose of 0.7 mg/kg. In some embodiments, the GC therapy is administered at a dose of 0.8 mg/kg. In some embodiments, the GC therapy is administered at a dose of 0.9 mg/kg. In some embodiments, the GC therapy is administered at a dose of 1 mg/kg. In some embodiments, the GC therapy is administered at a dose of 1.1 mg/kg. In some embodiments, the GC therapy is administered at a dose of 1.2 mg/kg. In some embodiments, the GC therapy is administered at a dose of 1.3 mg/kg. In some embodiments, the GC therapy is administered at a dose of 1.4 mg/kg. In some embodiments, the GC therapy is administered at a dose of 1.5 mg/kg. In some embodiments, the GC therapy is administered at a dose of 1.6 mg/kg. In some embodiments, the GC therapy is administered at a dose of 1.7 mg/kg. In some embodiments, the GC therapy is administered at a dose of 1.8 mg/kg. In some embodiments, the GC therapy is administered at a dose of 1.9 mg/kg. In some embodiments, the GC therapy is administered at a dose of 2.0 mg/kg.

In some embodiments, the GC therapy is administered at a dose of 20-60 mg/day prednisone or equivalent. In some embodiments, the GC therapy is administered at a dose of 10-100 mg/day prednisone or equivalent. In some embodiments, the GC therapy is administered at a dose of about 1-70 mg/day, about 5-70 mg/day, about 10-70 mg/day, about 15-70 mg/day, about 20-70 mg/day, about 25-70 mg/day, about 30-70 mg/day, about 35-70 mg/day, about 40-70 mg/day prednisone or equivalent. In some embodiments, the GC therapy is administered at a dose of about 1-60 mg/day, about 5-60 mg/day, about 10-60 mg/day, about 15-60 mg/day, about 20-60 mg/day, about 25-60 mg/day, about 30-60 mg/day, about 35-60 mg/day, about 40-60 mg/day prednisone or equivalent.

In some embodiments, the GC therapy is administered at a dose of about 1-150 mg/day, about 5-150 mg/day, about 10-150 mg/day, about 15-150 mg/day, about 20-150 mg/day, about 25-150 mg/day, about 30-150 mg/day, about 35-150 mg/day, about 40-150 mg/day, about 45-150 mg/day, about 50-150 mg/day, about 55-150 mg/day, about 60-150 mg/day, about 65-150 mg/day, about 70-150 mg/day, about 75-150 mg/day, about 80-150 mg/day, about 90-150 mg/day, or about 100-150 mg/day, prednisone or equivalent. In some embodiments, the GC therapy is administered at a dose of about 5-120 mg/day, about 5-110 mg/day, about 10-90 mg/day, about 15-100 mg/day, about 20-100 mg/day, about 25-100 mg/day, about 30-100 mg/day, about 35-100 mg/day, about 40-100 mg/day, about 45-100 mg/day, about 50-100 mg/day, about 55-100 mg/day, about 60-100 mg/day, about 65-100 mg/day, about 70-100 mg/day, about 75-100 mg/day, about 80-100 mg/day, or about 90-100 mg/day prednisone or equivalent.

In some embodiments, the GC therapy is administered at a dose of up to about 150 mg/day, up to about 120 mg/day, up to about 110 mg/day, up to about 100 mg/day, up to about 90 mg/day, up to about 80 mg/day, up to about 70 mg/day, up to about 60 mg/day, up to about 50 mg/day, up to about 40 mg/day, up to about 30 mg/day, up to about 20 mg/day, up to about 15 mg/day, up to about 10 mg/day, up to about 5 mg/day, or up to about 1 mg/day prednisone or equivalent.

In some embodiments, the GC therapy is administered at a dose of 0.1-1 mg/kg/day, a dose of 0.1-0.8 mg/kg/day, a dose of 0.1-0.7 mg/kg/day, a dose of 0.1-0.6 mg/kg/day, a dose of 0.1-0.5 mg/kg/day, a dose of 0.1-0.4 mg/kg/day, a dose of 0.1-0.3 mg/kg/day, a dose of 0.1-0.2 mg/kg/day or a dose of 0.05-0.1 mg/kg/day prednisone or equivalent.

In some embodiments, the GC therapy is administered at a dose of up to 1 mg/kg/day prednisone or equivalent. In some embodiments, the GC therapy is administered at a dose of up to 0.9 mg/kg/day, a dose of up to 0.8 mg/kg/day, a dose of up to 0.7 mg/kg/day, a dose of up to 0.6 mg/kg/day, a dose of up to 0.5 mg/kg/day, a dose of up to 0.4 mg/kg/day, a dose of up to 0.3 mg/kg/day, a dose of up to 0.2 mg/kg/day or dose of up to 0.1 mg/kg/day prednisone or equivalent.

In some embodiments, the GC therapy is administered at a high dose of prednisone or equivalent. In some embodiments, the patient has been administered a high dose of GC therapy and is unlikely to respond to any treatment therapy. In some embodiments, the patient has not responded to prior therapies before administration of the anti-CD19 antibody (e.g., obexelimab).

In some embodiments, the GC therapy continues during the treatment with obexelimab. In some embodiments, the GC therapy is tapered during treatment with obexelimab. In some embodiments, the GC therapy is tapered prior to treatment with obexelimab. In some embodiments, the GC therapy is tapered to complete discontinuation. In some embodiments, obexelimab is administered in combination with a GC therapy.

### EXAMPLES

### Example 1: Administration of Obexelimab for Treating wAIHA

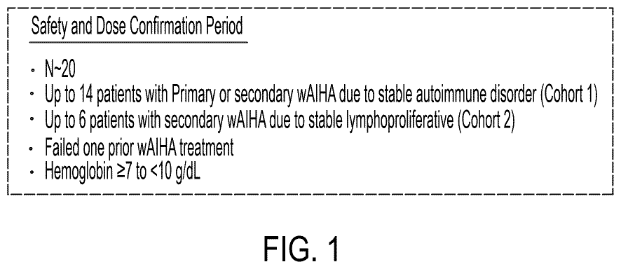

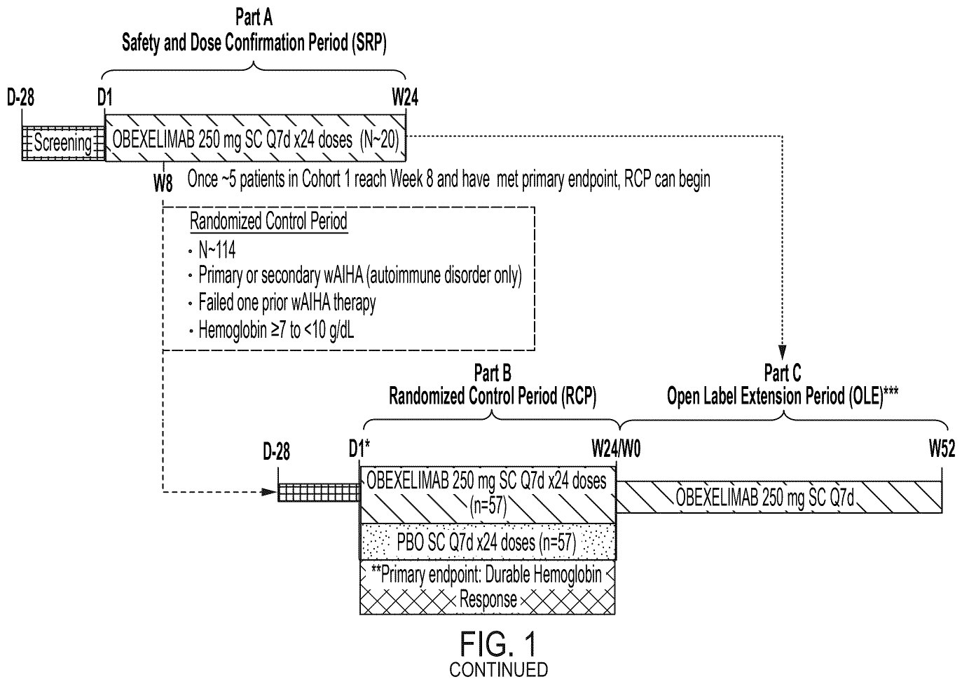

This Example describes exemplary patient selection criteria for administration of an anti-CD19 antibody (e.g., obexelimab) to patients enrolled in a Phase 2/3, multicenter, randomized, double-blind, placebo-controlled study. As shown in FIG. 1, the study included an open-label safety and dose confirmation run-in period (SRP), to evaluate the safety and efficacy of obexelimab in patients with wAIHA. Patients selected for the randomized-control period (RCP) using the inclusion and exclusion criteria described below will receive placebo formulation or obexelimab.

Placebo without active substance will be supplied also as a solution for SC injection. The placebo formulation is: 2.35 mg/mL sodium acetate trihydrate, 0.17 mg/mL acetic acid (at density 1.053 g/mL), 30 mg/mL L-proline, 0.1 mg/mL polysorbate 80, 115 mg/mL Dextran-40 at pH 5.5. The SC formulation of the placebo is a sterile liquid product supplied in single-use glass vials. Each 2-mL glass vial is filled with 1.2 mL of placebo. The single-use glass vial is masked to make it indistinguishable from obexelimab. Table 2 shows the dosage regimen and formulation of Obexelimab.

During the Randomized control period (RCP), patients will undergo assessments for efficacy, safety, PK, pharmacodynamics (PD), and immunogenicity at study visits specified in the Schedule of Assessments (SoA). Adverse events (AE), serious adverse events (SAE) and treatment-emergent adverse events (TEAEs) or clinically significant safety laboratory abnormalities will be evaluated.

### Inclusion Criteria

Patients are selected for treatment based on the following criteria:
1. Males and females≥18 years of age.
2. Diagnosed with wAIHA for at least 3 months and currently receiving treatment for wAIHA or have previously received treatment for wAIHA (treatment-naive patients are not eligible)
3. Diagnosis of primary or secondary wAIHA as documented by a positive DAT specific for anti-IgG or anti-IgA
4. Failed at least 1 prior wAIHA treatment regimen, including steroids, rituximab, azathioprine, cyclophosphamide, cyclosporine, mycophenolate mofetil, danazol, vincristine, erythropoiesis-stimulating agents, or splenectomy (folate, iron, or other supplements do not fulfill this criterion)
5. If on prednisone/prednisolone, the dose may not exceed 20 mg/day and must have been stable for at least 4 weeks prior to randomization and remain stable throughout the SRP and RCP
6. If receiving immunosuppressants, must have been on a stable dose for at least 12 weeks prior to randomization and remain on a stable dose throughout the SRP and RCP. Allowed concomitant immunosuppressants are azathioprine, mycophenolate mofetil/mycophenolic acid, cyclosporine, and cyclophosphamide
7. Hgb≥7 to <10 g/dL
8. At least one sign or symptom of anemia as assessed by the investigator at baseline
9. Screening platelet count≥50,000 mm³
10. Screening neutrophil count≥1,000 mm³
11. Screening serum albumin and serum calcium concentrations within the normal range
12. Screening total serum IgG of ≥600 mg/dL
13. Screening creatine kinase value<2×ULN
14. Patients with a history of splenectomy must be at least 4 months post resection prior to randomization and must be vaccinated as per country-specific immunization schedules
15. Patients with autoimmune disorders (e.g., systemic lupus erythematosus, rheumatoid arthritis) may be eligible if they are receiving stable treatment (no changes in disease-related concomitant medications), and the severity of disease has been stable for at least 4 months prior to randomization
16. For the SRP only (Part A), patients with LPDs (Cohort 2) may be eligible if they are receiving stable treatment (no changes in concomitant disease-related medications), the severity of disease has been stable for at least 4 months prior to randomization, and, in the opinion of the Investigator, they are unlikely to require chemotherapy or mAb therapy during the study

### Exclusion Criteria

Patients may be selected for treatment with obexelimab based on the exclusion criteria:
1. Have cold antibody AIHA, cold agglutinin syndrome, mixed type (i.e., warm and cold) AIHA, or paroxysmal cold hemoglobinuria
2. Have any other associated cause of hereditary or acquired hemolytic anemia
3. For the RCP only (Part B), patients with secondary wAIHA not due to autoimmune disorders, including LPDs
4. Received a transfusion within 2 weeks prior to randomization
5. Use of B cell-depleting, B cell-targeted, or other biologic immunomodulatory agents within the 6 months prior to randomization. Patients who received B cell-targeted therapy within 6 to 12 months prior to randomization must have a B cell count at screening that is within the laboratory reference range, as measured by the central laboratory.
6. Received IV Ig or epoetin alfa within 6 weeks prior to randomization. The patient may be re-screened after the exclusionary period of 6 weeks has passed
7. Receiving more than 2 concomitant medications for the treatment of wAIHA, excluding vitamins or other supplements, at the time of screening
8. Received an investigational treatment or direct medical intervention in another clinical study within 12 weeks or <5 half-lives of the investigational treatment, whichever is shorter, prior to screening
9. Received live vaccine or live therapeutic infectious agent within the 6 weeks prior to randomization
10. Evidence of active tuberculosis (TB) or at high risk for TB based on at least one of the following:
a. History of active TB or latent TB, unless completion of treatment according to local guidelines is documented
b. Positive, indeterminate, or invalid interferon-gamma (IFNγ) release assay results at screening, unless treatment is documented. Patients with an indeterminate test result can repeat the test once either centrally or locally, but if the repeat test is also indeterminate, the patient is excluded
c. Signs of symptoms that could represent active TB
d. Chest radiograph, computed tomography scan, or magnetic resonance imaging that suggests possible diagnosis of TB
11. History or evidence of a clinically unstable/uncontrolled disorder, condition, or disease (including, but not limited to, cardiopulmonary, oncologic, renal, hepatic, metabolic, hematologic, psychiatric, active infection), that, in the opinion of the investigator, would pose a risk to patient safety or interfere with the study evaluations, procedures, or completion
12. Known allergy to monoclonal antibody therapy
13. Known hypersensitivity to dextran or components of dextran
14. Active infection (e.g., pneumonia, biliary tract infection, diverticulitis, Clostridium difficile infection) that requires parenteral or oral anti-infectives and/or hospitalization, and/or is assessed as serious/clinically significant by the investigator, within 8 weeks prior to screening. Patients may be re-screened after the 8-week exclusionary period has passed
15. Chronic infection (e.g., bronchiectasis, chronic osteomyelitis, chronic pyelonephritis) or requiring chronic treatment with anti-infectives (e.g., antibiotics, antivirals)
16. Confirmed or suspected clinical immunodeficiency syndrome not related to treatment of wAIHA, or has a family history of congenital or hereditary immunodeficiency, unless confirmed absent in the patient
17. Acute hepatitis B infection (hepatitis B surface antigen-positive), active hepatitis C virus (HCV), or HIV infection. Patients may be excluded from treatmentif they have a positive test for active hepatitis B through detection of hepatitis B surface antigen. In Japan, patients will be excluded if there is detection of
(a) hepatitis B surface antigen or
(b) hepatitis B surface antibody or
(c) hepatitis B core antibody. Patients with a history of HCV may be excluded in the study unless there is documentation of a negative HCV ribonucleic acid level in the serum at 12 weeks or longer after the completion of HCV therapy.

Patients receiving obexelimab experience significantly improved HgB levels, compared to patients receiving placebo.

### Example 2: Efficacy Analyses and Endpoints

This Example demonstrates efficacy analysis of the clinical trial discussed in described in Example 1. The primary efficacy endpoint is the proportion of patients with a Hgb response.

Samples are collected at baseline and at weekly study visits. Assessment parameters include evaluating patients who achieve a durable Hgb response (defined as Hgb≥10 g/dL and/or ≥2 g/dL increase from baseline) and measuring LDH levels. Blood chemistry and hematology panels are assessed to determine Hgb levels in response to treatment with obexelimab. If a patient demonstrates reappearance of prior sign/symptoms or new signs/symptoms of wAIHA, a physical examination, imaging, and/or biochemical parameters are obtained.

Secondary assessment parameters include evaluating quality of life improvements using, for example, change from Baseline to week 24 in FACIT-F Score, EQ-5D-5L index score, Physician's Global Assessment of Change Disease Activity, Patient's Global Impression of Change in Daily Activity Impact, and/or Patient's Global Impression of Change in Fatigue Severity.

Additional assessment parameters include change from baseline through week 24 over time in the following:
Reduction in Circulating absolute T, B, and NK cell count
Reduction in Ig levels and ratios (e.g., IgG, IgM, IgA, IgE)
Increase in CD19 target receptor occupancy
Decrease in reticulocyte count
Decrease in LDH
Increase in Haptoglobin
Reduction in indirect bilirubin (unconjugated bilirubin)

Secondary assessments of disease activity in the SRP include the change from baseline in FACIT-F score through week 24. proportion of patients with no use of blood transfusion or GC rescue therapy through week 24, cumulative dose of GC rescue therapy through week 24, proportion of patients with a durable Hgb response.

In Part A (for safety and dose confirmation run-in period): Approximately 20 patients are enrolled. Patients are categorized into 2 cohorts. Cohort 1 will consist of patients with primary wAIHA or secondary wAIHA due to auto-immune disorders (e.g., systemic lupus erythematous). Cohort 2 will consist of patients with secondary wAIHA due to a lymphoproliferative disease (LPD). Outcomes from this part will provide preliminary safety, tolerability, PK/PD, and efficacy data in a similar wAIHA population as the population intended to be enrolled in the randomized control period. The randomized control period (Part B) is initiated once all 14 patients with primary wAIHA or secondary wAIHA due to underlying autoimmune disorder (Cohort 1) have reached Week 8-12 (or withdrawn from the study) and at least 5 have met the primary endpoint, in the absence of TEAEs Grade 4 or above no significant safety events in Cohort 1, and PK and PD data that are comparable to PK/PD data in other clinical studies and PK modelling of obexelimab. Outcomes from patients with secondary wAIHA enrolled into Cohort 2 and will provide preliminary data on a broad wAIHA population.

The primary analysis of Hgb response is performed in Cohort 1 and is based on the point estimate of Hgb response rate on or after Week 8 and the corresponding 90% exact confidence interval. Demonstrating that the lower confidence bound is greater than 15% for the Hgb response rate is considered clinically meaningful and represents the likelihood a patient who has failed prior wAIHA treatment would achieve the endpoint without any additional treatment.

The order of testing primary and key secondary efficacy endpoints is as follows:
1. The proportion of patients who achieve a durable Hgb response (defined as Hgb≥10 g/dL and ≥2 g/dL increase from Baseline on at least 3 of 4 consecutive available visits), at the earliest on or after Week 12, with no use of blood transfusion or GC rescue therapy prior to attaining durable response (primary endpoint)
2. Change in FACIT-F score from Baseline to Week 24 (key secondary endpoint)
3. Proportion of patients with no use of blood transfusion or GC rescue therapy through Week 24 (key secondary endpoint)
4. Cumulative dose of GC rescue therapy through Week 24 (key secondary endpoint)
5. Proportion of patients with no use of blood transfusions through Week 24 (key secondary endpoint)

### OTHER EMBODIMENTS

While a number of embodiments of this invention are described herein, the present disclosure and examples may be altered to provide other methods and compositions of this invention. Therefore, it will be appreciated that the scope of this invention is to be defined by the appended claims in addition to the specific embodiments that have been represented by way of example. All references cited herein are hereby incorporated by reference.

1. A method of treating autoimmune hemolytic anemia (AIHA), comprising administering obexelimab subcutaneously to a human patient at a dose of 250 mg once a week.

2. The method of claim 1, wherein the patient has a Hgb level of ≥7 to <10 g/dL.

3. The method of claim 1 or 2, wherein the patient has been diagnosed with warm autoimmune hemolytic anemia (wAIHA).

4. The method of any one of the preceding claims, wherein the patient has at least one sign or symptom of anemia.

5. The method of any one of the preceding claims, wherein the patient also has failed at least 1 prior wAIHA treatment regimen.

6. The method of any one of claim 5, wherein the prior wAIHA treatment regimen is GC or immunosuppression therapy.

7. The method of claim 5, wherein the failure of the prior wAIHA treatment regimen comprises reduction in Hgb of ≥1 g/dL.

8. The method of claim 5, wherein the failure of the prior wAIHA treatment regimen comprises an increase in LDH of ≥1.5×upper limit of normal (ULN).

9. The method of any one of the preceding claims, wherein obexelimab is administered concurrently with a GC therapy.

10. The method of claim 9, wherein the GC therapy is administered at a dose of 1-1.5 mg/kg/day prednisone or equivalent.

11. The method of any one of the preceding claims, wherein the patient maintains a Hgb level of ≥7 g/dL following administration of obexelimab.

12. The method of any one of the preceding claims, wherein the patient maintains a Hgb level of ≥7 g/dL, ≥8 g/dL, ≥9 g/dL or ≥10 g/dL.

13. The method of any one of the preceding claims, wherein the patient achieves a Hgb≥10 g/dL.

14. The method of any one of the preceding claims, wherein the patient achieves a Hgb≥2 g/dL increase compared to Hgb levels prior to treatment with obexelimab.

15. The method of any one of the preceding claims, wherein obexelimab is administered for a time period sufficient to improve, stabilize or reduce one or more symptoms of wAIHA relative to a control.

16. The method of any one of the preceding claims, wherein the patient achieves improvement in Hgb after at least 4, 5, 6, 7, 8, 9, 10, 11, 12 weeks of obexelimab administration.

17. The method of any one of the preceding claims, wherein the patient achieves improvement in FACIT-F score compared to the FACIT-F score prior to treatment.

18. The method of any one of the preceding claims, wherein the patient achieves improvement in EQ-5D-5L index score compared to the EQ-5D-5L index score prior to treatment.

19. The method of any one of the preceding claims, wherein the patient achieves one or more of the following:
a) Decrease in Circulating absolute T, B, and NK cell count,
b) Decrease in Ig levels and ratios (e.g., IgG, IgM, IgA, IgE),
c) Increase in CD19 target receptor occupancy,
d) Decrease in reticulocyte count,
e) Decrease in LDH,
f) Increase in Haptoglobin,
g) Decrease in indirect bilirubin (unconjugated bilirubin) following administration obexelimab.

20. The method of any one of the preceding claims, wherein the human patient is relapsed or refractory to rituximab.

21. The method of any one of the preceding claims, wherein the patient 18 years of age or older.

22. The method of any one of the preceding claims, wherein the patient does not have cold autoimmune hemolytic amenia or CAD.

23. The method of any one of the preceding claims, wherein the patient does not have mixed type autoimmune hemolytic amenia.

24. The method of any one of the preceding claims, wherein the patient does not have Paroxysmal cold hemoglobinuria (PCH).

25. The method of any one of the preceding claims, wherein obexelimab is administered in a liquid formulation comprising 125 mg/mL obexelimab, 2.35 mg/mL sodium acetate trihydrate, 0.17 mg/mL acetic acid, 30 mg/mL L-proline, 0.1 mg/mL polysorbate 80 at pH 5.5.

26. The method of claim 25, wherein obexelimab is administered as 2×1 mL injections or 1×2 mL injection.

27. The method of claim 25 or 26, wherein obexelimab is administered using a prefilled syringe or autoinjector.


SEQUENCE LISTING (12 sequences)

SEQ ID NO: 1 - protein, 556 aa, Homo sapiens [sequence omitted]

SEQ ID NO: 2 - protein, 16 aa, synthetic construct
RSSKSLQNVNGNTYLY

SEQ ID NO: 3 - protein, 7 aa, synthetic construct
RMSNLNS

SEQ ID NO: 4 - protein, 9 aa, synthetic construct
MQHLEYPIT

SEQ ID NO: 5 - protein, 5 aa, synthetic construct
SYVMH

SEQ ID NO: 6 - protein, 14 aa, synthetic construct
WIGYINPYNDGTKY

SEQ ID NO: 7 - protein, 12 aa, synthetic construct
GTYYYGTRVFDY

SEQ ID NO: 8 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 12 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS